(19) United States
(12) Patent Application Publication
(10) Pub. No.: US 2009/0198069 A1
(43) Pub. Date: Aug. 6, 2009

(54) METAL COMPLEX, LIGHT-EMITTING DEVICE AND DISPLAY

(75) Inventors: Keisuke Umakoshi, Nagasaki-shi (JP); Takashi Kojima, Nagasaki-shi (JP); Seiji Akatsu, Nagasaki-shi (JP); Masayoshi Onishi, Nagasaki-shi (JP); Shoji Ishizaka, Sapporo-shi (JP); Noboru Kitamura, Sapporo-shi (JP)

Correspondence Address: DITTHAVONG MORI & STEINER, P.C., 918 Prince St., Alexandria, VA, 22314, US

(73) Assignee: NAGASAKI UNIVERSITY, NATIONAL UNIVERSITY CORPORATI, Nagasaki-shi (JP)

(21) Appl. No.: 11/909,686

(22) PCT Filed: Mar. 24, 2006
(86) PCT No.: PCT/JP2006/306792

§ 371 Date: Mar. 13, 2009

(30) Foreign Application Priority Data
Mar. 25, 2005 (JP) 2005-089421
Sep. 20, 2005 (JP) 2005-272942
Dec. 27, 2005 (JP) 2005-375999

(51) Int. Cl. C07F 15/00

(52) U.S. Cl. 548/101

(57) ABSTRACT

An object of the present invention is to provide a novel metal complex. A metal complex having a composition of [(Pt^{II})₂(M^{I})₄(L)₈], where M^{I} is H⁺, Ag^{I}, Au^{I} or Cu^{I}, and L is a compound represented by the following chemical formula. A metal complex having a composition of [(Pt^{II})₂(M^{I})₄(X)₂(L)₆], where M^{I} is Ag^{I}, Au^{I} or Cu^{I}, X is Cl⁻, Br⁻ or I⁻, and L is a compound represented by the following chemical formula. In the formula, R¹, R² and R³ are independently a hydrogen atom, a chlorine atom, a bromine atom, an iodine atom, a hydroxyl group, a phenyl group, a trifluoromethylphenyl group, a pentafluorophenyl group, a naphthyl group, a methyl group, an ethyl group, an i-propyl group, a t-butyl group, a trifluoromethyl group, a hydroxymethyl group or a hydroxyethyl group. At least one of R¹, R² and R³ in the formula is not a hydrogen atom.

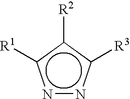

### TECHNICAL FIELD

The present invention relates to a metal complex. The present also relates to a light-emitting device including the metal complex in a light-emitting layer. The present further relates to a display including the light-emitting device as a component.

### BACKGROUND ART

Recently, organic EL devices have attracted attention as light-emitting displays alternative to liquid crystal displays. Organic EL devices of the related art utilize emission (fluorescence) from a singlet excited state. In this case, a local maximum emission efficiency is 25% based on a principle of an organic EL phenomenon, and therefore emission is extremely insufficient.

Phosphorescence generated from a triplet excited state has attracted most attention recently as a method of increasing emission efficiency (see Non-Patent Document 1, for example). In this case, the emission efficiency may be 100% in theory.

Many Pt(II) complexes having diimine or terpyridine and their derivatives exhibit emission which are assigned to MLCT or MMLCT, and photochemical properties of these compounds have attracted much interest (See Non-Patent Document 2, for example). Polynuclear Cu(I) and Au(I) complexes of pyrazolate and its derivatives are also known to exhibit emission (see Non-Patent Document 3, for example). Accordingly, when a molecule is synthesized with Pt(II) ions and Cu(I) ions, Ag(I) ions or Au(I) ions and these metal ions are bridged by pyrazolate or its derivatives, it is promising to produce a new molecule having emission properties by a synergetic effect of different metal ions.

In development of a novel metal complex based on this idea, a mixed metal complex [Pd₂Ag₄(μ-dmpz)₈] having two Pd(II) ions and four Ag(I) ions bridged by 3,5-dimethylpyrazolate ligands (see Non-Patent Document 4) is known as an analogous compound; however, emission properties of this compound have never been reported. The present inventors also have already synthesized a mixed metal complex [Pt₂Ag₄(μ-pz)₈] having Pt(II) ions and Ag(I) ions bridged by pyrazolate ligands without substituent groups (see Non-Patent Document 5); however, this compound does not show emission.

Furthermore, in development of displays for commercialization, there is an increasing demand for a novel metal complex used as a dopant having improved thermal stability, volatility, film-forming properties during deposition, solubility in various solvents, emission intensity, color purity, and stability when applying a potential.
[Non-Patent Document 1] M. A. Baldo, S. Lamansky, P. E. Burrows, M. E. Thompson, S. R. Forrest, Appl. Phys. Lett. 1999, 75, 4-6.
[Non-Patent Document 2] S.-W. Lai, C.-M. Che, Topics in Current Chemistry, 2004, 241 (Transition Metal and Rare Earth Compounds III), 27-63.
[Non-Patent Document 3] H. V. R. Dias, H. V. K. Diyabalanage, M. G. Eldabaja, O. Elbjeirami, M. A. Rawashdeh-Omary, M. A. Omary, J. Am. Chem. Soc. 2005, 127, 7489-7501.
[Non-Patent Document 4] G. A. Ardizzoia, G. La Monica, S. Cenini, M. Moret, N. Masciocchi, J. Chem. Soc. Dalton Trans. 1996, 1351-1357.
[Non-Patent Document 5] K. Umakoshi, Y. Yamauchi, K. Nakamiya, T. Kojima, M. Yamasaki, H. Kawano, M. Onishi, Inorg. Chem. 2003, 42, 3907-3916.

### DISCLOSURE OF THE INVENTION

The present invention has been attempted to solve such a problem. An object of the present invention is to provide a novel metal complex.

Another object of the present invention is to provide a novel light-emitting device including the metal complex in a light-emitting layer.

Still another object of the present invention is to provide a novel display including the light-emitting device as a component.

In order to solve the aforementioned problem and achieve the object of the present invention, a first metal complex of the present invention includes a composition of [(Pt^{II})₂(M^{I})₄(L)₈], where (M^{I})₄ are protons, silver ions, copper ions or gold ions, and (L)₈ are each or a combination of any of compounds represented by the following chemical formula 1:

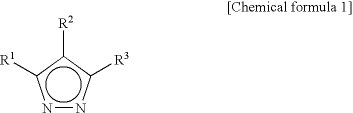

where R¹, R² and R³ are independently a hydrogen atom, a chlorine atom, a bromine atom, an iodine atom, a hydroxyl group, a phenyl group, a trifluoromethylphenyl group, a pentafluorophenyl group, a naphthyl group, a methyl group, an ethyl group, an i-propyl group, a t-butyl group, a trifluoromethyl group, a hydroxymethyl group or a hydroxyethyl group, provided that at least one of R¹, R² and R³ is not a hydrogen atom.

A first light-emitting device of the present invention includes a light-emitting layer, which includes the first metal complex of the present invention.

A first display of the present invention includes a light-emitting device as a component, and the light-emitting device including a light-emitting layer, which includes the first metal complex of the present invention.

To solve the aforementioned problem and achieve the object of the present invention, a second metal complex of the present invention includes a composition of [(Pt^{II})₂(M^{I})₄(X)₂(L)₆], where (M^{I})₄ are silver ions, copper ions or gold ions, (X)₂ are chloride, bromide ions or iodide ions, and (L)₆ are one or a combination of any of compounds represented by the following chemical formula 2:

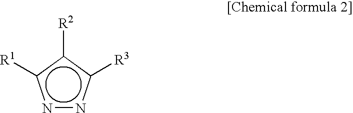

where R¹, R² and R³ are independently a hydrogen atom, a chlorine atom, a bromine atom, an iodine atom, a hydroxyl group, a phenyl group, a trifluoromethylphenyl group, a pentafluorophenyl group, a naphthyl group, a methyl group, an ethyl group, an i-propyl group, a t-butyl group, a trifluoromethyl group, a hydroxymethyl group or a hydroxyethyl group, provided that at least one of R¹, R² and R³ is not a hydrogen atom.

A second light-emitting device of the present invention includes a light-emitting layer that includes the second metal complex of the present invention.

A second display of the present invention includes a light-emitting device as a component that includes the light-emitting device having a light-emitting layer, which includes the second metal complex of the present invention.

The present invention has the following effect.

The first metal complex of and the second metal complex of the present invention may provide a novel metal complex.

The first light-emitting device and second light-emitting device of the present invention may provide a novel light-emitting device.

The first light-emitting device and the second light-emitting device of the present invention may provide a novel light-emitting device.

### BRIEF DESCRIPTION OF THE DRAWINGS

FIG. 1 is a cross-sectional view showing an example of a light-emitting device of the present invention.

FIG. 2 is an ORTEP diagram showing a molecular structure of [{Pt(dmpz)₂(dmpzH)₂}₂].

FIG. 3 is an ORTEP diagram showing a molecular structure of [Pt₂Ag₄(μ-dmpz)₈].

FIG. 4 is an ORTEP diagram showing a molecular structure of [{Pt(3-Mepz)₂(3-MepzH)₂}₂].

FIG. 5 is an ORTEP diagram showing a molecular structure of Pt(3-^{t}Bupz)₂(3-^{t}BupzH)₂}.

FIG. 6 is an ORTEP diagram showing a molecular structure of [Pt₂Ag₄(μ-3-^{t}Bupz)₈]. Methyl carbon atoms in t-butyl groups are omitted for clarity.

FIG. 7 is an ORTEP diagram showing a molecular structure of [Pt₂Cu₄(μ-3-^{t}Bupz)₈]. Methyl carbon atoms in t-butyl groups are omitted for clarity.

FIG. 8 is an ORTEP diagram showing a molecular structure of [Pt₂Ag₄(μ-dmpz)₈].

FIG. 9 is an ORTEP diagram showing a molecular structure of [PtCl(dppz)(dppzH)₂].

FIG. 10 is an ORTEP diagram showing a molecular structure of [Pt₂Ag₄(μ-Cl)₂(μ-dppz)₆].

### BEST MODE FOR CARRYING OUT THE INVENTION

A best mode for carrying out the present invention will be described below.

First, a first metal complex of the present invention will be described.

The first metal complex of the present invention includes a composition represented by the following formula:

[(Pt^{II})₂(M^{I})₄(L)₈]

where (M^{I})₄ are protons, silver ions, copper ions or gold ions, and (L)₈ are each or a combination of any of compounds represented by the chemical formula 1.

In the chemical formula 1, R¹, R² and R³ are independently a hydrogen atom, a chlorine atom, a bromine atom, an iodine atom, a hydroxyl group, a phenyl group, a trifluoromethylphenyl group, a pentafluorophenyl group, a naphthyl group, a methyl group, an ethyl group, an i-propyl group, a t-butyl group, a trifluoromethyl group, a hydroxymethyl group or a hydroxyethyl group.

At least one of R¹, R² and R³ is not a hydrogen atom, that is, at least one of R¹, R² and R³ is a substituent group. This is because the metal complex in which R¹, R² and R³ are all hydrogen atoms, namely the metal complex without substituent groups, does not exhibit emission.

In the next part, the synthesis of the first metal complex of the present invention will be described. Here, dmpzH denotes 3,5-dimethylpyrazole, and dmpz denotes a monovalent anion in which a proton is dissociated from 3,5-dimethylpyrazole.

First, the synthesis of [{Pt(dmpz)₂(dmpzH)₂}₂] will be described as an example of the first metal complex of the present invention. This metal complex is a first metal complex where M^{I} is a proton, and the complex can be regarded as a precursor for the synthesis of the complex [Pt₂M^{I}₄(μ-dmpz)₈] (M^{I}=Ag^{I}, Cu^{I}, Au^{I}).

The complex [{Pt(dmpz)₂(dmpzH)₂}₂] may be synthesized by the following procedure, for example.

The reaction of [PtCl₂(C₂H₅CN)₂] with dmpzH gives [Pt(dmpzH)₄]Cl₂.

[Pt(dmpzH)₄]Cl₂ further reacts with dmpzH in the presence of KOH to afford [{Pt(dmpz)₂(dmpzH)₂}₂].

The method for synthesizing [Pt(dmpzH)₄]Cl₂ is not limited to the aforementioned method. There are also the following two other synthetic methods.

One synthetic method: [PtCl₂(C₂H₅CN)₂] is suspended in water, methanol or ethanol. An excess amount of dmpzH is added to the suspension, and the mixture is refluxed for one hour. The solution is allowed to cool and then concentrated under reduced pressure. The addition of acetone or diethyl ether into the residue gives the precipitate of [Pt(dmpzH)₄]Cl₂. The precipitated [Pt(dmpzH)₄]Cl₂ is collected, washed with diethyl ether, and then dried in vacuo.

Another synthetic method: K₂[PtCl₄] is dissolved in acidic water. Four equivalents of dmpzH are added to the solution, and the mixture is refluxed for six hours. The solution is allowed to cool and then concentrated in vacuo, and acetone is added to the residue to precipitate [Pt(dmpzH)₄]Cl₂. The precipitated [Pt(dmpzH)₄]Cl₂ is collected, washed with diethyl ether, and then dried in vacuo.

The synthetic method of [{Pt(dmpz)₂(dmpzH)₂}₂] is not limited to the aforementioned method. There is also the following other synthetic method.

[PtCl₂(C₂H₅CN)₂] is dissolved in acetonitrile or propionitrile. Two equivalents of triethylamine and four equivalents of dmpzH are added to the solution, and the mixture is refluxed for six hours. The solution is cooled and allowed to evaporate under air, thereby precipitating [{Pt(dmpz)₂(dmpzH)₂}₂].

The precipitate of [{Pt(dmpz)₂(dmpzH)₂}₂] is collected, washed with a small amount of acetonitrile or propionitrile, and then dried in vacuo. The addition of methanol to the filtrate further gives the precipitate of [{Pt(dmpz)₂(dmpzH)₂}₂]. This precipitate is also collected in the same manner.

Next, the synthesis of [Pt₂Ag₄(μ-dmpz)₈] will be described as an example of the first metal complex of the present invention.

The reaction of [{Pt(dmpz)₂(dmpzH)₂}₂], which is synthesized as a precursor by the above mentioned procedure, with AgBF₄ in the presence of triethylamine affords [Pt₂Ag₄(μ-dmpz)₈].

The method for synthesizing [Pt₂Ag₄(μ-dmpz)₈] is not limited to the aforementioned method. There are also the following two other synthetic methods. It is noted here that the aforementioned precursor is not used in the following methods.

One synthetic method: [PtCl₂(C₂H₅CN)₂] is dissolved in acetonitrile or propionitrile. Four equivalents of AgBF₄ or AgPF₆ are added to the solution, and the mixture is refluxed for one hour. The solution is allowed to cool, and then the precipitate of AgCl is filtered off. Four equivalents of triethylamine and four equivalents of dmpzH are added to the filtrate and the mixture is refluxed for further two hours. The solution is filtered in hot state and the filtrate is slowly evaporated under air, thereby precipitating [Pt₂Ag₄(μ-dmpz)₈]. The precipitate of [Pt₂Ag₄(μ-dmpz)₈] is collected, washed with a small amount of acetonitrile or propionitrile, and then dried in vacuo. The addition of methanol to the filtrate further gives the precipitate of [Pt₂Ag₄(μ-dmpz)₈]. This precipitate is also collected in the same manner.

Another synthetic method: [Pt(dmpzH)₄]Cl₂ is dissolved in methanol or ethanol. Four equivalents of AgBF₄ or AgPF₆ are added to the solution, and the mixture is stirred for an hour at room temperature. The resulted AgCl is filtered off. Four equivalents of triethylamine are added to the filtrate, and the mixture is refluxed for further one hour. The precipitate of [Pt₂Ag₄(μ-dmpz)₈] is collected, washed with a small amount of methanol or ethanol, and then dried in vacuo.

Furthermore, there is also the following method for synthesizing [Pt₂Ag₄(μ-dmpz)₈] without using triethylamine.

[{Pt(dmpz)₂(dmpzH)₂}₂] is suspended in acetonitrile. Four equivalents of AgBF₄ or AgPF₆ are added to the suspension, and the mixture is stirred for six hours. The precipitated [Pt₂Ag₄(μ-dmpz)₈] is collected, washed with a small amount of acetonitrile, and then dried in vacuo.

Next, the synthetic method of [{Pt(3-Mepz)₂(3-MepzH)₂}₂] will be described as an example of the first metal complex of the present invention. This metal complex is also a first metal complex where M^{I} is a proton, and the metal complex can be regarded as a precursor for the synthesis of [Pt₂M^{I}₄(3-Mepz)₈] (M^{I}=Ag^{I}, Cu^{I}, Au^{I}). Here, 3-MepzH denotes 3-methylpyrazole, and 3-Mepz represents a monovalent anion in which a proton is dissociated from 3-methylpyrazole.

The complex [{Pt(3-Mepz)₂(3-MepzH)₂}₂] may be synthesized by the following procedure, for example.

The reaction of [PtCl₂(C₂H₅CN)₂] with 3-MepzH gives a white solid.

The white solid further reacts with 3-MepzH in the presence of KOH to afford [{Pt(3-Mepz)₂ (3-MepzH)₂}₂].

The method for synthesizing [{Pt(3-Mepz)₂(3-MepzH)₂}₂] is not limited to the aforementioned method. There is also the following other synthetic method.

[PtCl₂(C₂H₅CN)₂] is dissolved in acetonitrile or propionitrile. Two equivalents of triethylamine and four equivalents of 3-MepzH are added to the solution, and the mixture is refluxed for six hours. The solution is cooled and allowed to evaporate under air, thereby precipitating [{Pt(3-Mepz)₂(3-MepzH)₂}₂]. The precipitate of [{Pt(3-Mepz)₂(3-MepzH)₂}₂] is collected, washed with a small amount of acetonitrile or propionitrile, and then dried in vacuo. The addition of methanol to the filtrate further gives the precipitate of [{Pt(3-Mepz)₂(3-MepzH)₂}₂]. This precipitate is also collected in the same manner.

Next, the synthetic method of [Pt₂Ag₄(μ-3-Mepz)₈] will be described as an example of the first metal complex of the present invention.

The reaction of metal complex [{Pt(3-Mepz)₂(3-MepzH)₂}₂], synthesized above as a precursor, with AgBF₄ in the presence of triethylamine affords [Pt₂Ag₄(μ-3-Mepz)₈].

The method for synthesizing [Pt₂Ag₄(μ-3-Mepz)₈] is not limited to the aforementioned method. There is also the following other synthetic method. It is noted here that the aforementioned precursor is not used in the following method.

[PtCl₂(C₂H₅CN)₂] is dissolved in acetonitrile or propionitrile. Four equivalents of AgBF₄ or AgPF₆ are added to the solution, and the mixture is refluxed for one hour. The solution is allowed to cool, and then the precipitated AgCl is filtered off. Four equivalents of triethylamine and four equivalents of 3-MepzH are added to the filtrate, and the mixture is refluxed for further two hours. The solution is filtered when it is still hot. The slow evaporation of the filtrate under air gives the precipitate of [Pt₂Ag₄(μ-3-Mepz)₈]. The obtained [Pt₂Ag₄(μ-3-Mepz)₈] is collected, washed with a small amount of acetonitrile or propionitrile, and then dried in vacuo. The addition of methanol to the filtrate further gives precipitate of [Pt₂Ag₄ (μ-3-Mepz)₈]. This precipitate is also collected in the same manner.

Next, the synthetic method of [Pt(3-^{t}Bupz)₂(3-^{t}BupzH)₂] will be described as an example of a precursor for the first metal complex of the present invention. Here, 3-^{t}BupzH denotes 3-t-butylpyrazole, and 3-^{t}Bupz denotes a monovalent anion in which a proton is dissociated from 3-t-butylpyrazole.

This complex [Pt(3-^{t}Bupz)₂(3-^{t}BupzH)₂] may be synthesized by the following procedure, for example.

The reaction of [PtCl₂(C₂H₅CN)₂] with 3-^{t}BupzH gives [Pt(3-^{t}BupzH)₄]Cl₂.

The treatment of [Pt(3-^{t}BupzH)₄]Cl₂ with KOH affords [Pt(3-^{t}Bupz)₂(3-^{t}BupzH)₂].

The synthetic method [Pt(3-^{t}Bupz)₂(3-^{t}BupzH)₂] is not limited to the aforementioned method. There is also the following other synthetic method.

[PtCl₂(C₂H₅CN)₂] is dissolved in acetonitrile or propionitrile. Two equivalents of triethylamine and four equivalents of 3-^{t}BupzH are added to the solution, and the mixture is refluxed for six hours. The solution is cooled and allowed to evaporate under air, thereby precipitating [Pt(3-^{t}Bupz)₂(3-^{t}BupzH)₂].

The precipitate of [Pt(3-^{t}Bupz)₂(3-^{t}BupzH)₂] is collected, washed with a small amount of acetonitrile or propionitrile, and then dried in vacuo. The addition of methanol to the filtrate further gives the precipitate of [Pt(3-^{t}Bupz)₂(3-^{t}BupzH)₂]. This precipitate is also collected in the same manner.

Next, the synthetic method of [Pt₂Ag₄(μ-3-^{t}Bupz)₈] will be described as an example of the first metal complex of the present invention.

The reaction of the precursor [Pt(3-^{t}Bupz)₂(3-^{t}BupzH)₂] with AgBF₄ in the presence of triethylamine affords [Pt₂Ag₄(μ-3-^{t}Bupz)₈].

The method for synthesizing [Pt₂Ag₄(μ-3-^{t}Bupz)₈] is not limited to the aforementioned method. There is also the following other synthetic method.

[PtCl₂(C₂H₅CN)₂] is dissolved in acetonitrile or propionitrile. Four equivalents of AgBF₄ or AgPF₆ are added to the solution, and the mixture is refluxed for one hour. The solution is allowed to cool, and then the precipitated AgCl is filtered off. Four equivalents of triethylamine and four equivalents of 3-^{t}BupzH are added to the filtrate, and the mixture is refluxed for further two hours. The solution is filtered in hot state, and the filtrate is allowed to evaporate under air to give the precipitate of [Pt₂Ag₄(μ-3-^{t}Bupz)₈]. The precipitate is collected, washed with a small amount of acetonitrile or propionitrile, and then dried in vacuo. The addition of methanol to the filtrate further gives the precipitate of [Pt₂Ag₄(μ-3-^{t}Bupz)₈]. This precipitate is also collected in the same manner.

Next, the synthetic method of [Pt₂Cu₄(μ-dmpz)₈] will be described as an example of the first metal complex of the present invention.

The reaction of the precursor complex [{Pt(dmpz)₂(dmpzH)₂}₂] with [Cu(CH₃CN)₄]BF₄ in the presence of triethylamine affords [Pt₂Cu₄(μ-dmpz)₈].

The method for synthesizing [Pt₂Cu₄(μ-dmpz)₈] is not limited to the aforementioned method. There is also the following other synthetic method.

[PtCl₂(C₂H₅CN)₂] is dissolved in acetonitrile or propionitrile. Four equivalents of [Cu(CH₃CN)₄]BF₄ are added to the solution, and the mixture is refluxed for one hour. The solution is allowed to cool, and then the precipitated CuCl is filtered off. Four equivalents of triethylamine and four equivalents of dmpzH are added to the filtrate, and the mixture is refluxed for further two hours. The solution is filtered in hot state and the filtrate is allowed to evaporate under air to give the precipitate of [Pt₂Cu₄(μ-dmpz)₈]. The precipitate is collected, washed with a small amount of acetonitrile or propionitrile, and then dried in vacuo. The addition of methanol to the filtrate further gives the precipitate of [Pt₂Cu₄(μ-dmpz)₈]. This precipitate is also collected in the same manner.

Next, the synthetic method of [Pt₂Cu₄(μ-3-^{t}Bupz)₈] will be described as an example of the first metal complex of the present invention.

The reaction of precursor complex [Pt(3-^{t}Bupz)₂(3-^{t}BupzH)₂] with [Cu(CH₃CN)₄]BF₄ in the presence of triethylamine affords [Pt₂Cu₄(μ-3-^{t}Bupz)₈].

The synthetic method of [Pt₂Cu₄(μ-3-^{t}Bupz)₈] is not limited to the aforementioned method. There is also the following other method.

[PtCl₂(C₂H₅CN)₂] is dissolved in acetonitrile or propionitrile. Four equivalents of [Cu(CH₃CN)₄]BF₄ are added to the solution, and the mixture is refluxed for one hour. The solution is allowed to cool, and then the precipitated CuCl is filtered off. Four equivalents of triethylamine and four equivalents of 3-^{t}BupzH are added to the filtrate, and the mixture is refluxed for further two hours. The solution is filtered in hot state and the filtrate is allowed to evaporate under air to give precipitate of [Pt₂Cu₄(μ-3-^{t}Bupz)₈]. The precipitate is collected, washed with a small amount of acetonitrile or propionitrile, and then dried in vacuo. The addition of methanol to the filtrate further gives the precipitate of [Pt₂Cu₄(μ-3-^{t}Bupz)₈]. This precipitate is also collected in the same manner.

In the next part, the second metal complex of the present invention will be described.

The second metal complex of the present invention includes a composition represented by the following formula:

[(Pt^{II})₂(M^{I})₄(X)₂(L)₆]

where (M^{I})₄ are silver ions, copper ions or gold ions, and (L)8 are each or a combination of any of compounds represented by the chemical formula 2.

At least one of R¹, R² and R³ is not a hydrogen atom, that is, at least one of R¹, R² and R³ is a substituent group. This is because the metal complex, where R¹, R² and R³ are all hydrogen atoms, that is, the metal complex without substituent group does not exhibit emission.

The synthetic methods for the second metal complex of the present invention will be described below. In the following, dppzH denotes 3,5-diphenylpyrazole, and dppz denotes a monovalent anion in which a proton is dissociated from 3,5-diphenylpyrazole.

At first, the synthetic method of [PtCl(dppz)(dppzH)₂] will be described as an example of a precursor complex for the second metal complex of the present invention.

This precursor complex [PtCl(dppz)(dppzH)₂] may be synthesized by the following procedure, for example.

The reaction of [PtCl₂(C₂H₅CN)₂] with dppzH, and the successive treatment of resulting white yellow precipitate with KOH gives [PtCl(dppz)(dppzH)₂]. [PtBr(dppz)(dppzH)₂] and [PtI(dppz)(dppzH)₂] may be synthesized similarly.

The synthetic method of [PtX(dppz)(dppzH)₂] (X═Cl⁻, Br⁻ or I⁻) is not limited to the aforementioned method. There is also the following other synthetic method.

[PtX₂(C₂H₅CN)₂] is suspended in water, methanol or ethanol. An excess amount of dppzH is added to the suspension, and the mixture is refluxed for one hour. The solution is allowed to cool and then concentrated under reduced pressure, and acetone or diethyl ether is added to the residue to form a precipitate. The formed precipitate is treated with a base such as NaOH or KOH, and the resulting solid is washed with methanol and water and then dried in vacuo.

Next, the synthetic method of [Pt₂Ag₄(μ-Cl)₂(μ-dppz)₆] will be described as an example of the second metal complex of the present invention.

The reaction of the precursor complex [PtCl(dppz)(dppzH)₂] with AgBF₄ in the presence of triethylamine [Pt₂Ag₄(μ-Cl)₂(μ-dppz)₆].

The synthetic method of [Pt₂Ag₄(μ-Cl)₂(μ-dppz)₆] is not limited to the aforementioned method. There is also the following other synthetic method.

[PtCl₂(C₂H₅CN)₂] is dissolved in acetonitrile or propionitrile Four equivalents of AgBF₄ or AgPF₆ are added to the solution, and the mixture is refluxed for one hour. The solution is allowed to cool, and then the precipitated AgCl is filtered off. Four equivalents of triethylamine and four equivalents of dppzH are added to the filtrate, and the mixture is refluxed for further two hours. The solution is filtered in hot state and the filtrate is allowed to evaporate under air to give the precipitate of [Pt₂Ag₄(μ-Cl)₂(μ-dppz)₆]. The precipitate is collected, washed with a small amount of acetonitrile or propionitrile, and then dried in vacuo. The addition of methanol to the filtrate further gives the precipitate of [Pt₂Ag₄(μ-Cl)₂(μ-dppz)₆]. This precipitate is also collected in the same manner.

There is another synthetic method without using triethylamine.

[PtCl(dppz)(dppzH)₂] is suspended in acetonitrile. Four equivalents of AgBF₄ or AgPF₆ are added to the suspension, and the mixture is stirred for six hours. The resulted precipitate of [Pt₂Ag₄(μ-Cl)₂(μ-dppz)₆] is collected, washed with a small amount of acetonitrile, and then dried in vacuo.

In the next part, the use of the metal complexes of the present invention will be described. The metal complexes can be used as luminescent agents contained in a light-emitting layer of a light-emitting device such as an organic EL device. However, the metal complexes may not be only used as luminescent agents. The metal complexes can also be used as sensors for organic molecules or gas molecules, antitumor agents, or paint that is usually colorless and transparent but emit light only upon exposure to UV radiation, for example.

Next, a light-emitting device including such metal complexes in a light-emitting layer will be described.

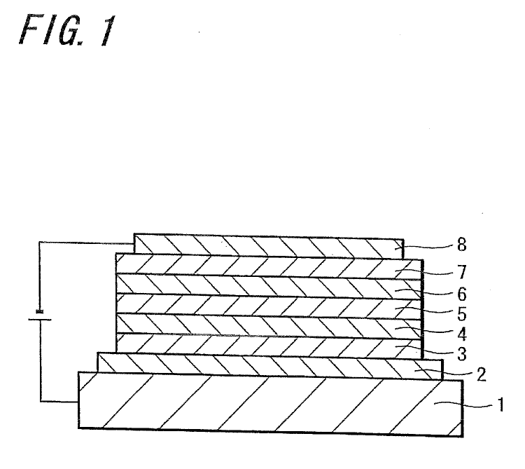

FIG. 1 is a cross-sectional view showing an example of the light-emitting device of the present invention. A substrate 1 is formed of a transparent material such as glass. An anode 2 is formed on the substrate 1. A hole injection layer 3, a hole transport layer 4, a light-emitting layer 5, an electron transport layer 6 and an electron injection layer 7 are formed on the anode 2. A cathode 8 is formed on the electron injection layer 7.

The light-emitting device of the present invention is not limited to the aforementioned five-layer light-emitting device. Alternatively, the light-emitting device may be a four-layer light-emitting device in which the electron transport layer is omitted from the five-layer light-emitting device. The light-emitting device may also be a three-layer light-emitting device in which the hole injection layer and the electron injection layer are omitted from the five-layer light-emitting device. The light-emitting device may also be a two-layer light-emitting device having one layer used as both a light-emitting layer and an electron transport layer of the three-layer light-emitting device. The light-emitting device may also be a single-layer light-emitting device having only a light-emitting layer formed between an anode and a cathode.

The light-emitting device in which the aforementioned metal complexes may be advantageously used is essentially a light-emitting device including metal complexes having light-emitting ability, and is usually mainly used as a stacked light-emitting device including an anode of applying positive voltage, a cathode of applying negative voltage, a hole injection/transport layer of injecting and transporting holes from the anode, an electron injection/transport layer of injecting and transporting electrons from the cathode, and a light-emitting layer of recombining the holes with the electrons to output light. These metal complexes have significant light-emitting ability and are therefore extremely useful as host luminescent agents in the light-emitting device. Furthermore, when a slight amount of these metal complexes is doped with a hole injection/transport layer material, an electron injection/transport layer material, or another host luminescent agent including a metal complex having 8-quinolinol as a ligand such as tris(8-hydroxyquinolinato)aluminum, these metal complexes function as guest luminescent agents to improve their emission efficiency and emission spectra. Therefore, in a light-emitting device including one or a plurality of such materials as essential elements, these metal complexes may be extremely advantageously used alone or in combination with another luminescent agents such as dicyanomethylene (DCM), coumarin, perylene or rubrene or a hole injection/transport layer material and/or an electron injection/transport layer material, for example. In a stacked light-emitting device, when luminescent agents also have hole injection/transport ability or electron injection/transport ability, a hole injection/transport layer or an electron injection/transport layer may be omitted, or when one of a hole injection/transport layer and an electron injection/transport layer functions as the other, the hole injection/transport layer or the electron injection/transport layer may be omitted, respectively.

These metal complexes may be used for both a single-layer light-emitting device and a stacked light-emitting device. An operation of a light-emitting device essentially includes a process of injecting electrons and holes from electrodes, a process of transferring the electrons and the holes in a solid, a process of recombining the electrons with the holes to produce a triplet exciton, and a process of allowing the exciton to emit light. These processes are essentially not different between a single-layer light-emitting device and a stacked light-emitting device. However, a stacked light-emitting device may generally provide desired performance more easily as compared with a single-layer light-emitting device. While in the single-layer light-emitting device, characteristics of the four processes may be improved only by changing the molecular structure of a luminescent agent, in the stacked light-emitting device, functions required for each process may be shared by a plurality of materials and each material may be independently optimized. Thus, desired performance may be more easily achieved in a case where the metal complexes are formed in the stacked light-emitting device than in a case where the metal complexes are formed in the single-layer light-emitting device.

The aforementioned light-emitting device may be used in a display. Specifically, a display including the light-emitting device as a component may include the aforementioned metal complex in a light-emitting layer of the light-emitting device.

The present invention is not limited to the aforementioned best mode for carrying out the present invention. Obviously, various other embodiments can be provided without departing from the gist of the present invention.

### EXAMPLES

Examples of the present invention will be specifically described. However, it should be noted that the present invention is not limited to these examples.

### Example 1

As one of the first metal complexes of the present invention, [{Pt(dmpz)₂(dmpzH)₂}₂] was synthesized as a precursor of [Pt₂M₄(μ-dmpz)₈] (M=Cu, Ag, Au), and [Pt₂Ag₄(μ-dmpz)₈] was synthesized by using this precursor.

Details of the synthetic method of the metal complexes will be described below.

First, [Pt(dmpzH)₄]Cl₂ was synthesized.

Specifically, a solution of dmpzH (1155 mg, 12.0 mmol) in toluene (40 ml) was added to a suspension of [PtCl₂(C₂H₅CN)₂] (1130 mg, 3.0 mmol) in toluene (20 ml), and the mixture was refluxed for three hours under an Ar atmosphere. The precipitated white solid was collected, sequentially washed with toluene, hexane and diethyl ether, and then dried in vacuo. The yield was 1905 mg (97 5%). The synthetic method of [Pt(dmpzH)₄]Cl₂ is shown in chemical reaction formula 1.

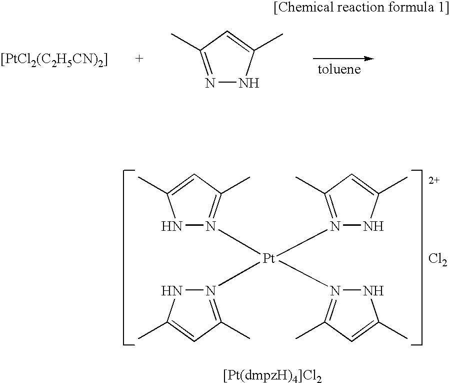

The product was identified by the IR and ¹H NMR spectra.

The infrared frequencies are as follows.

### IR (KBr): 3121 (m), 3068 (s), 2927 (s), 2849 (s), 2765 (s), 1580 (s), 1420 (m), 1297 (m), 1195 (w), 1150 (w), 1075 (w), 806 (m)

The ¹H NMR data are summarized in Table 1.

TABLE 1
¹H NMR of [Pt(dmpzH)₄]Cl₂ (in CD₃OD, 300 MHz)

| δ (ppm) | Shape (J/Hz) | Int. | Assign. |
|---|---|---|---|
| 6.12 | s | 1 | H4 of dmpzH |
| 2.29 | s | 3 | 5-CH₂ of dmpzH |
| 1.94 | s | 3 | 3-CH₃ of dmpzH |

Next, the precursor complex [{Pt(dmpz)₂(dmpzH)₂}₂] was synthesized.

Specifically, [Pt(dmpzH)₄]Cl₂ (101 mg, 0.16 mmol) was dissolved into methanol (6 ml). A methanol solution (1 ml) containing KOH (17 mg, 0.31 mmol) and dmpzH (30 mg, 0.31 mmol) was added dropwise to the methanol solution of [Pt(dmpzH)₄]Cl₂ with stirring at room temperature, so that a white precipitate was formed immediately. After stirring for further one hour, the formed white solid was collected, sequentially washed with methanol and water, and then dried in vacuo. The yield was 88 mg (98%). The synthetic method of [{Pt(dmpz)₂(dmpzH)₂}₂] is shown in chemical reaction formula 2.

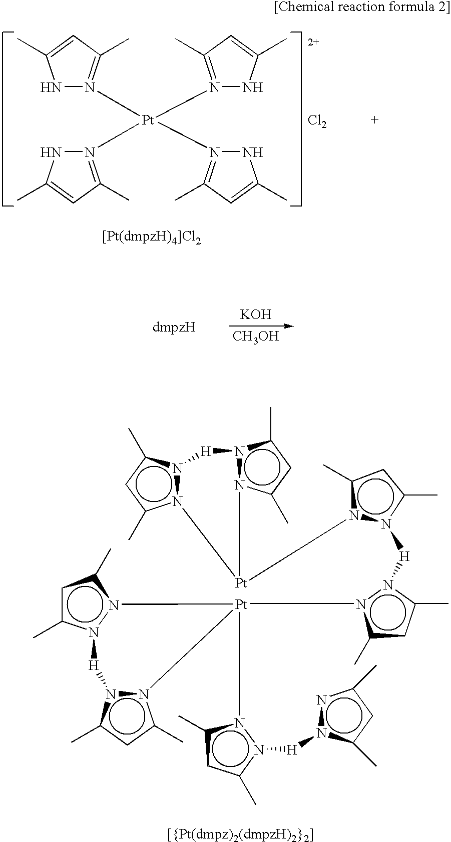

This metal complex was recrystallized from chloroform/methanol to yield a single crystal.

This complex exhibits bright pale orange luminescence in the solid state upon exposure to UV radiation. However, the luminescence is weaker in the solution.

With regard to solubility in a solvent, the compound is extremely highly soluble in chloroform and dichloromethane, highly soluble in benzene and toluene, moderately soluble in acetonitrile and diethyl ether, and not soluble in acetone, methanol and water.

This compound is decomposed at around 270° C.

The product was identified by the IR and ¹H NMR spectra.

The infrared frequencies are as follows.

IR (KBr): 3069 (w), 2923 (s), 2853 (m), 1869 (m, br), 1581 (m), 1531 (m), 1419 (s) 1342 (m), 1147 (w), 1151 (w), 765 (m)

The ¹H NMR data are summarized in Table 2.

TABLE 2
¹H NMR of [{Pt(dmpz)₂(dmpzH)₂}₂] (in CDCl₃, TMS, 300 MHz)

| δ (ppm) | Shape (J/Hz) | Int. | Assign. |
|---|---|---|---|
| 18.38 | s, br | 1 | NH |
| 5.45 | s | 2 | H4 of dmpzH |
| 1.74 | s | 6 | 6-CH₃ of dmpzH |
| 1.63 | s | 6 | 3-CH₃ of dmpzH |

The following discussion was made with reference to spectral data of [{Pd(dmpz)₂(dmpzH)₂}₂] reported by Ardizzoia et al. in the aforementioned Non-Patent Document 4. A broad absorption band at 1868 cm⁻¹ is the most significant characteristic in the IR spectrum of [{Pt(dmpz)₂(dmpzH)₂}₂].

In [{Pd(dmpz)₂(dmpzH)₂}₂], a similar band is observed at 1850 cm⁻¹ (Nujol mull), and this band is attributed to a ν(N—H...N) stretching vibration. A whole spectral shape of [{Pt(dmpz)₂(dmpzH)₂}₂] is very similar to that of [{Pd(dmpz)₂(dmpzH)₂}₂].

A characteristic signal is observed at 18.38 ppm in the ¹H NMR spectrum of [{Pt(dmpz)₂(dmpzH)₂}₂]. Since the chemical shift value of [{Pt(dmpz)₂(dmpzH)₂}₂] is extremely close to that at 18.1 ppm originating from the NH proton participating in the intermolecular hydrogen bonding in [{Pd(dmpz)₂(dmpzH)₂}₂] (in CD₂Cl₂), this compound may have a similar dimer structure.

The results of elemental analysis of the product are shown in Table 3 by comparison with calculated values.

TABLE 3
Elemental Analysis of [{Pt(dmpz)₂(dmpzH)₂}₂]

| | Calc. | Found |
|---|---|---|
| C(%) | 41.59 | 41.39 |
| H(%) | 5.24 | 5.21 |
| N(%) | 19.40 | 19.38 |

Next, the metal complex [Pt₂Ag₄ (μ-dmpz)₈] was synthesized from the precursor complex [{Pt(dmpz)₂(dmpzH)₂}₂].

Specifically, a solution of triethylamine (18 mg, 0.18 mmol) in acetonitrile (10 ml) and a solution of AgBF₄ (35 mg, 0.18 mmol) in acetonitrile (10 ml) were added to a suspension of the precursor complex [{Pt(dmpz)₂(dmpzH)₂}₂] (52 mg, 0.045 mmol) in acetonitrile (20 ml) at room temperature, and the mixture was stirred for two hours. After completion of the reaction, the white solid containing a small amount of silver salt was collected, washed with acetonitrile, and dried in vacuo (when it was difficult to collect the solid, the reaction solution was concentrated to dryness and the residue was recrystallized from chloroform/methanol). The yield was 57 mg (81%). The synthetic method of [Pt₂Ag₄(μ-dmpz)₈] is shown in chemical reaction formula 3.

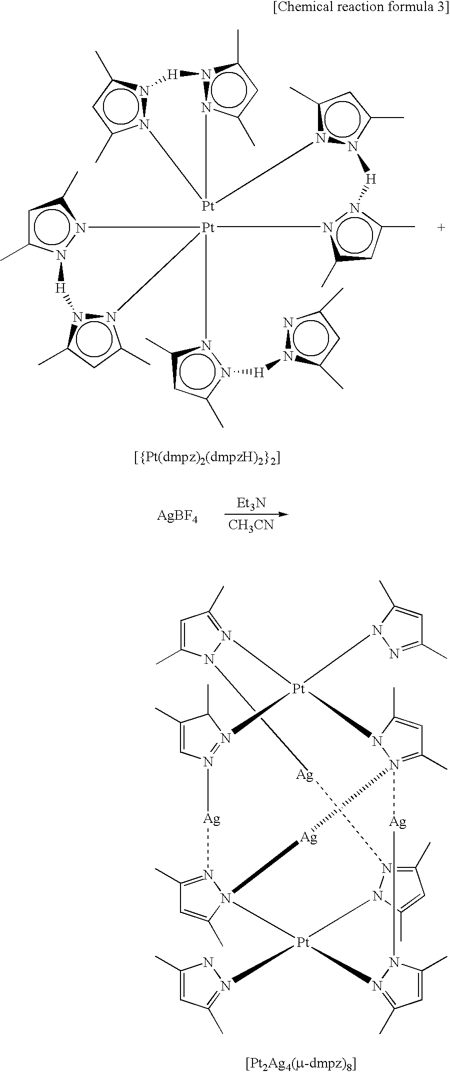

This metal complex was recrystallized from chloroform/methanol to yield a single crystal.

This complex exhibits bright sky-blue in the solid state and green-blue luminescence in solution, respectively, upon exposure to UV radiation.

With regard to solubility in a solvent, the complex is extremely highly soluble in chloroform and dichloromethane, highly soluble in benzene and toluene, moderately soluble in acetonitrile and hexane, and not soluble in acetone and methanol.

The compound has a melting point of 300° C. or more.

The product was identified by the IR and ¹H NMR spectra.

The infrared frequencies are as follows.

### IR (KBr): 3113 (w), 2971 (w), 2919 (m), 2856 (w), 1578 (w), 1529 (s), 1420 (s), 1351 (m), 1158 (w), 1084 (w), 1050 (w), 762 (s)

The ¹H NMR data are summarized in Table 4.

TABLE 4
¹H NMR of [Pt₂Ag₄(μ-dmpz)₈] (in CDCl₃, TMS, 300 MHZ)

| δ (ppm) | Shape (J/Hz) | Int. | Assign. |
|---|---|---|---|
| 5.70 | d, br | 1 | H4 of dmpzH |
| 1.90 | s | 3 | CH₂ of dmpzH |
| 1.82 | s | 3 | CH₃ of dmpzH |

Since the broad absorption band at 1868 cm⁻¹ in the IR spectrum and the signal at 18.38 ppm in the ¹H NMR spectrum, which are characteristic to [{Pt(dmpz)₂(dmpzH)₂}₂], disappear in the corresponding spectra of [Pt₂Ag₄(μ-dmpz)₈], it is assumed that all H atoms participating in the N—H...N hydrogen bonding are replaced by Ag atoms in [Pt₂Ag₄(μ-dmpz)₈].

The results of elemental analysis of the product are shown in Table 5 in comparison with calculated values.

TABLE 5
Elemental Analysis of [Pt₂Ag₄(μ-dmpz)₈]

| | Calc. | Found |
|---|---|---|
| C(%) | 30.36 | 30.25 |
| H(%) | 3.57 | 3.52 |
| N(%) | 14.16 | 14.05 |

Structures of the precursor complex and the finally produced metal complex will be described, respectively.

Molecular structures of [{Pt(dmpz)₂(dmpzH)₂}₂] and [Pt₂Ag₄(μ-dmpz)₈] were determined by single crystal X-ray structural analysis. The crystallographic data of these complexes are shown in Table 6.

TABLE 6
Crystallographic data of
[{Pt(dmpz)₂(dmpzH)₂}₂] and [Pt₂Ag₄(μ-dmpz)₈]

| | [{Pt(dmpz)₂(dmpzH)₂}₂] | [Pt₂Ag₄(dmpz)₈] |
|---|---|---|
| empirical formula | C₄₀H₆₀N₁₆Pt₂ | C₄₀H₅₆N₁₆Ag₄Pt₂ |
| fw | 1155.20 | 1582.64 |
| τ, K | 296 | 296 |
| λ, Å | 0.71069 | 0.71069 |
| cryst syst | monoclinic | monoclinic |
| space group | C2/c (15) | C2/c (15) |
| a, Å | 17.285(1) | 20.831(2) |
| b, Å | 15.753(2) | 12.924(2) |
| c, Å | 17.4782 (3) | 19.4719(3) |
| α, deg | 90 | 90 |
| β, deg | 100.3796(4) | 105.1428(4) |
| γ, deg | 90 | 90 |
| v, Å³ | 4681.2(5) | 5060.3(8) |
| Z | 4 | 4 |
| ρcalcd, Mg tu⁻³ | 1.639 | 7.077 |
| μ(Mo K α), mm⁻¹ | 5.993 | 2.044 |
| no. of unique rf lns | 5204 (Rᵢₙₜ = 0.021) | 5667 (Rᵢₙₜ = 0.047) |
| data/restraints/params | 5204/0/270 | 4270/0/280 |
| final R Indices | R1 = 0.025 | R1 = 0.044 |
| [I > 2σ(I)] | | |
| R indices (all data) | R = 0.057, R_{w} = 0.069 | R = 0.087, R_{w} = 0.159 |
| GOF | 1.01 | 0.97 |

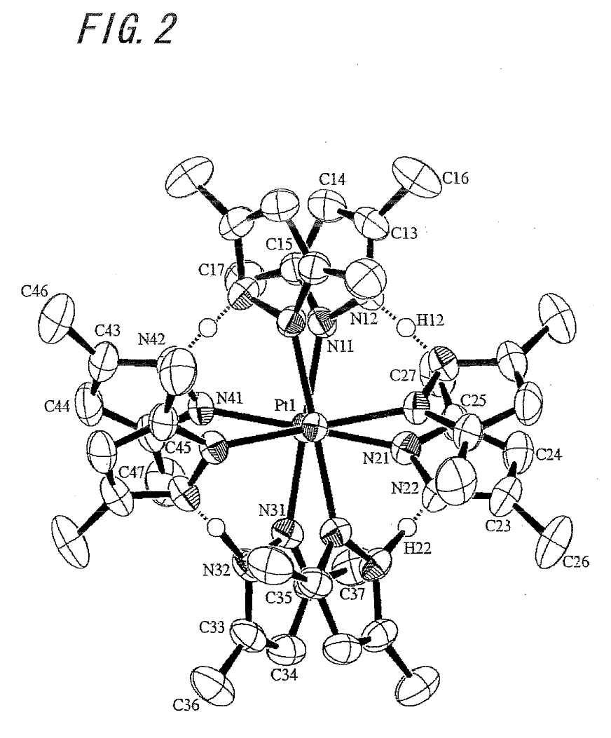

In the molecular structure of [{Pt(dmpz)₂(dmpzH)₂}₂], as shown in the ORTEP diagram of FIG. 2, two molecules of a mononuclear complex {Pt(dmpz)₂(dmpzH)₂} having two dmpz ligands and two dmpzH ligands coordinated to a Pt(II) ion are dimerized by forming strong N—H...N hydrogen bonds between dmpzH ligands of one molecule and dmpz ligands of the other molecule. The Pt—Pt distance in the dimer is 3.7205(3) Å.

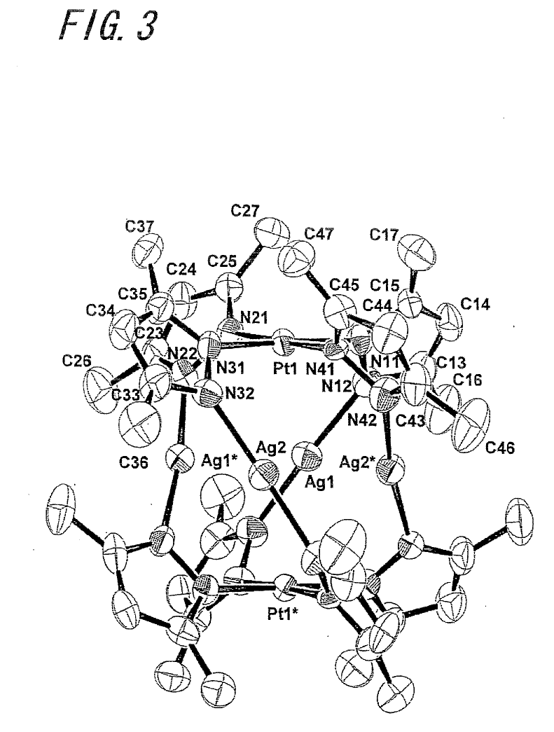

In the molecular structure of [Pt₂Ag₄(μ-dmpz)₈], four protons participating in the hydrogen bonding in [{Pt(dmpz)₂(dmpzH)₂}₂] are replaced by Ag⁺ ions, as shown in the ORTEP diagram of FIG. 3. [Pt₂Ag₄(μ-dmpz)₈] has an idealized four-fold axis passing through the two Pt atoms and two different sets of two-fold axes perpendicular to the four-fold rotation axis. The Pt...Pt distance in [Pt₂Ag₄(μ-dmpz)₈] is 5.1578(8) Å. The Pt...Ag distances are close to one another, ranging from 3.4514(7) to 3.5147(8) Å.

Next, photochemical properties of [{Pt(dmpz)₂(dmpzH)₂}₂] will be described.

Since the platinum complex [{Pt(dmpz)₂(dmpzH)₂}₂] was found to exhibit pale orange luminescence in the solid state upon exposure to UV radiation, the photochemical properties of the dimer complex were studied in detail.

A shoulder was observed at around 260 nm in the absorption spectrum. It is assumed that the shoulder originates from CT transition, because the molar extinction coefficient is about 10180 M⁻¹ cm⁻¹.

Emission properties of [{Pt(dmpz)₂(dmpzH)₂}₂] are shown in Table 7.

TABLE 7
Emission properties of [{Pt(dmpz)₂(dmpzH)₂}₂]

| Solvent | λ max/nm | φ | τ/ns | kᵣ/10³ s⁻¹ | kₙᵣ/10⁶ s⁻¹ |
|---|---|---|---|---|---|
| CH₂Cl₂(ε = 8.9) | ~600 | 0.001 | 210 | 4.8 | 4.8 |

On the other hand, broad emission having the local maximum at around 600 nm was observed in the emission spectrum. It is presumed that the absorption local maximum position approximately corresponds to a wavelength of emission color observed with the naked eye.

The emission quantum yield was determined by using a solution of [Ru(bpy)₃](PF₆)₂ (bpy=2,2′-bipyridine) in acetonitrile (Φ=0.061, under degassed conditions) as a reference (standard material). The sample solution was deoxygenated with a stream of argon prior to the measurement. The emission quantum yield is calculated from the equation (1) using an area integral S of each emission spectrum indicated by wavenumbers.

$$\begin{matrix}[ Equation 1 ] & \\ {Φ}_{x} = {Φ}_{ST} \times \frac{{S}_{x}}{{S}_{ST}} \times \frac{{A}_{ST}}{{A}_{x}} \times \frac{{n}_{ST}^{2}}{{n}_{x}^{2}} & ( 1 )\end{matrix}$$

In the equation (1) A and n denote an absorbance at an excitation wavelength and a refractive index of a solvent, respectively, and the subscript ST and X denote a standard material and measured sample.

The result shows that the emission quantum yield in dichloromethane was 0.001 and therefore the emission in the solution was extremely weak.

The excitation wavelength in the emission lifetime measurement was 266 nm. The emission lifetime was also as short as 210 ns.

The radiative deactivation rate constant (kᵣ) and the non-radiative deactivation rate constant (kₙᵣ) were calculated from measured values of the emission quantum yield and the emission lifetime. The results indicate that Kₙᵣ dominantly contributes to emission properties of the complex [{Pt(dmpz)₂(dmpzH)₂}₂].

Next, photochemical properties of [Pt₂Ag₄(μ-dmpz)₈] will be described.

Since this Pt—Ag mixed metal hexanuclear complex [Pt₂Ag₄(μ-dmpz)₈] was found to exhibit sky-blue luminescence in the solid state and green-blue luminescence in solution upon exposure to UV radiation, the photochemical properties of the this complex were studied in detail.

It is assumed that a broad absorption band observed at around 273 nm in the absorption spectrum originates from CT transition, because the spectrum does not have a vibrational structure and the molar extinction coefficient is 8640 M⁻¹ cm⁻¹.

Emission properties of [Pt₂Ag₄(μ-dmpz)₈] in three solvents (CH₂Cl₂, CHCl₃, and toluene) are listed in Table 8, respectively.

TABLE 8
Solvent dependence of emission properties of [Pt₂Ag₄(μ-dmpz)₈]

| Solvent | λ max/nm | φ | τ/μs | kᵣ/10⁴ s⁻¹ | kₙᵣ/10⁵ s⁻¹ |
|---|---|---|---|---|---|
| CH₂Cl₂(ε = 8.9) | 528 | 0.51 | 6.0 | 8.4 | 0.8 |
| CHCl₃(ε = 4.8) | 524 | 0.44 | 5.4 | 8.1 | 1.0 |
| Toluene (ε = 2.4) | 521 | 0.35 | 4.8 | 7.4 | 1.3 |

On the other hand, in the emission spectrum, broad emission characteristic to MLCT having the local maximum at around 528 nm was observed in dichloromethane, as shown in Table 8. The emission is presumably from a triplet state, because the Stokes shift is large and the emission lifetime at room temperature is 6.0 μs.

Solvent (dielectric constant) dependence was measured in order to verify that the emission is based on CT. A red shift of the emission maximum was observed in accordance with an increase of the dielectric constant of solvent.

Therefore, it is assumed that the emissive state for [Pt₂Ag₄(μ-dmpz)₈] might be assigned to ³MLCT state.

The emission quantum yield was determined by using a solution of 9,10-Diphenylanthlacene (DPA) in cyclohexane (Φ=0.91, under degassed conditions) as a reference (standard material). The sample solution was deoxygenated with a stream of argon prior to the measurement. The excitation wavelength was 335 nm. The emission quantum yield was 0.51 in dichloromethane. Thus this complex will exhibit extremely strong emission as a metal complex. This value is considerably higher than the emission quantum yield of 0.4 (λₘₐₓ=514 nm) of a fac-[Ir^{III}(ppy)₃] (ppy=2-phenylpyridinato), which is industrially used for an organic EL device (OLED). The emission quantum yield also depends on the solvent. Interestingly, the complex exhibits higher emission quantum yield in the solvent with higher dielectric constant, that is, when the complex exhibits emission at a longer wavelength, the emission quantum yield was high. This is contrary to the behavior common to general MLCT emission (Energy-Gap law) and is assumed to be one of the features in the photochemical properties of [Pt₂Ag₄(μ-dmpz)₈].

The excitation wavelength in the emission lifetime measurement was 355 nm. The emission decay curve may be analyzed by a single exponential function in any solvent. The result shows that, as in the case of emission quantum yield, the emission lifetime varies depending on the solvent and the lifetime is longer in emission at longer wavelength.

The complex [Pt₂Ag₄(μ-dmpz)₈] has a radiative deactivation rate constant (kᵣ) in the order of 10⁴ (s⁻¹) and a non-radiative deactivation rate constant (kₙᵣ) in the order of 10⁵ (s⁻¹), where the kₙᵣ contributes to emission properties of [Pt₂Ag₄(μ-dmpz)₈] slightly more significantly than the kₙᵣ. The kᵣ is almost constant but the kₙᵣ varies among the solvents. Furthermore, as in the case of emission quantum yield, the solvent dependence of the non-radiative deactivation rate constant (kₙᵣ) is contrary to the Energy-Gap law.

### Example 2

As one of the first metal complexes of the present invention, [{Pt(3-Mepz)₂(3-MepzH)₂}₂] was synthesized as a precursor of [Pt₂M₄(μ-3-Mepz)₈] (M=Cu, Ag, Au), and [Pt₂Ag₄(μ-3-Mepz)₈] was synthesized by using this precursor.

Details of the synthetic method of the metal complexes will be described below.

First, the precursor complex [{Pt(3-Mepz)₂(3-MepzH)₂}₂] was synthesized.

Specifically, a solution of 3-MepzH (66 mg, 0.8 mmol) in toluene (5 ml) was added to a suspension of [PtCl₂(C₂H₅CN)₂] (60 mg, 0.16 mmol) in toluene (5 ml), and the mixture was refluxed overnight under an Ar atmosphere (the mixture was changed from an yellow suspension to an yellow solution and further to a white suspension). The precipitated white solid was collected, sequentially washed with toluene, hexane and diethyl ether, and then dried in vacuo. The yield was 86 mg (0.14 mmol) (90%).

Next, a solution of KOH (17 mg, 0.31 mmol) and 3-MepzH (34 mg, 0.41 mmol) in methanol (1 ml) was added dropwise to a solution of the resulting white powder (98 mg, 0.16 mmol) in methanol (6 ml) with stirring at room temperature, so that a white precipitate was formed immediately. The reaction mixture was stirred for one hour and then concentrated by an evaporator. The resulting white solid was collected, sequentially washed with a small amount of methanol and water, and then dried in vacuo. The precursor complex [{Pt(3-Mepz)₂(3-MepzH)₂}₂] was obtained in this manner. The yield was 70 mg (0.067 mmol) (84%).

These reactions are shown in chemical reaction formula 4.

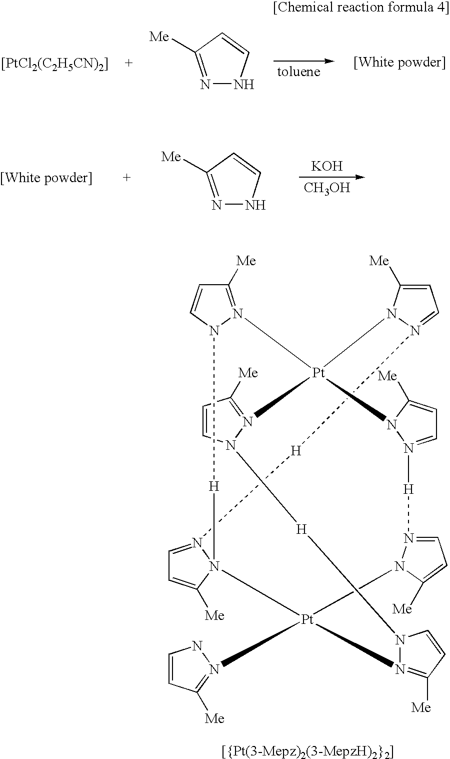

Both the white powder and [{Pt(3-Mepz)₂(3-MepzH)₂}₂] exhibit very weak orange luminescence in the solid state upon exposure to UV radiation.

Characteristics of the white powder are as follows.

With regard to solubility in a solvent, the compound is soluble in acetone and methanol.

The infrared frequencies are as follows.

IR (KBr): 3437 (br/s), 3045 (br/s), 2856 (br/s), 1577 (w), 1543 (m), 1486 (m), 1369 (s), 1282 (m), 1213 (s), 1142 (w), 1127 (w), 1080 (s), 778 (s), 608 (s), 416 (w), 321 (w)

Characteristics of [{Pt(3-Mepz)₂(3-MepzH)₂}₂] are as follows.

With regard to solubility in a solvent, [{Pt(3-Mepz)₂(3-MepzH)₂}₂] is readily soluble in chloroform and methylene chloride and soluble in benzene, toluene and diethyl ether.

The infrared frequencies are as follows

IR (KBr) 3106 (w), 2925 (w) 1865 (br/w), 1696 (w), 1508 (s), 1439 (m) 1356 (s) 1133 (m), 1033 (m), 854 (w), 751 (s)

The ¹H NMR data are summarized in Table 9.

TABLE 9
¹H NMR of [{Pt(3-Mepz)₂(3-MepzH)₂}₂] (CDCl₃,
TMS, 300 MHz)

| δ (ppm) | Shape (J/Hz) | Int. | Assighn. |
|---|---|---|---|
| 19.04 | s, br | 1 | NH |
| 6.62 | s | 2 | H5 of 3-MepzH |
| 5.67 | s | 2 | H4 of 3-MepzH |
| 1.67 | s | 6 | 3-CH₃ of 3-MepzH |

Next, the metal complex [Pt₂Ag₄(μ-3-Mepz)₈] was synthesized from the precursor complex [{Pt(3-Mepz)₂(3-MepzH)₂}₂].

Specifically, triethylamine (18 mg, 0.18 mmol) and a solution of AgBF₄ (41 mg, 0.21 mmol) in acetonitrile (10 ml) were sequentially added to a suspension of the precursor complex [{Pt(3-Mepz)₂(3-MepzH)₂}₂] (47 mg, 0.045 mmol) in acetonitrile (20 ml) at room temperature, and the mixture was stirred for two hours. The white solid was collected, washed with acetonitrile, and then dried in vacuo. The yield was 57 mg (0.039 mmol) (87%).

The synthetic method of [Pt₂Ag₄(μ-3-Mepz)₈] is shown in chemical reaction formula 5.

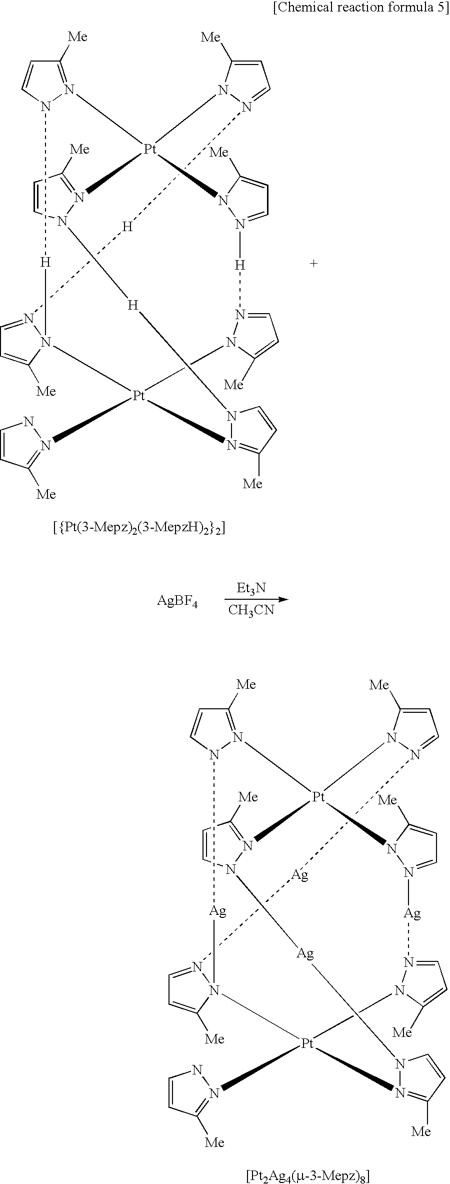

This complex exhibits bright sky-blue luminescence in the solid state and yellow-green luminescence in solution, respectively, upon exposure to UV radiation.

With regard to solubility in a solvent, the compound is soluble in chloroform and methylene chloride and slightly soluble in benzene, toluene and acetonitrile.

The product was identified by the IR and ¹H NMR spectra.

The infrared frequencies are as follows.

### IR (KBr): 3106 (w), 2923 (w), 1506 (s), 1480 (m), 1437 (m), 1356 (s), 1205 (m), 1129 (s), 1033 (m), 954 (m), 852 (w), 755 (s), 321 (m)

The ¹H NMR data are summarized in Table 10.

TABLE 10
¹H NMR of [Pt₂Ag₄(μ-3-Mepz)₈] (CDCl₃, TMS, 300 MHz)

| δ (ppm) | Shape (J/Hz) | Int. | Assighn. |
|---|---|---|---|
| 7.02 | s | 1 | H5 of 3-MepzH |
| 5.95 | s | 1 | H4 of 3-MepzH |
| 1.94 | s | 3 | 3-CH₃ of 3-MepzH |

The results of elemental analysis of the product are shown in Table 11 by comparison with calculated values.

TABLE 11
Elemental Analysis of [Pt₂Ag₄(μ-3-Mepz)₈]

| | Calc. | Found |
|---|---|---|
| C(%) | 26.14 | 26.39 |
| H(%) | 2.74 | 2.47 |
| N(%) | 15.24 | 15.18 |

### Example 3

A metal complex [Pt(3-^{t}Bupz)₂(3-^{t}BupzH)₂] was synthesized as a precursor complex, and [Pt₂Ag₄(μ-3-^{t}Bupz)₈], which was one of the first metal complex of the present invention, was synthesized by using this precursor complex.

Details of the synthetic method of the metal complexes will be described below.

First, [Pt(3-^{t}BupzH)₄]Cl₂ was synthesized.

Specifically, a solution 3-^{t}BupzH (99 mg, 0.8 mmol) in toluene (10 ml) was added to a suspension of [PtCl₂(C₂H₅CN)₂] (71 mg, 0.19 mmol) in toluene (5 ml), and the mixture was refluxed for four hours under an Ar atmosphere (the mixture was changed from an yellow suspension to an yellow solution). The solution was concentrated to dryness by an evaporator, and the white solid was sequentially washed with hexane and diethyl ether and dried in vacuo. The yield was 139 mg (0.18 mmol) (96%). The synthetic method of [Pt(3-^{t}BupzH)₄]Cl₂ is shown in chemical reaction formula 6.

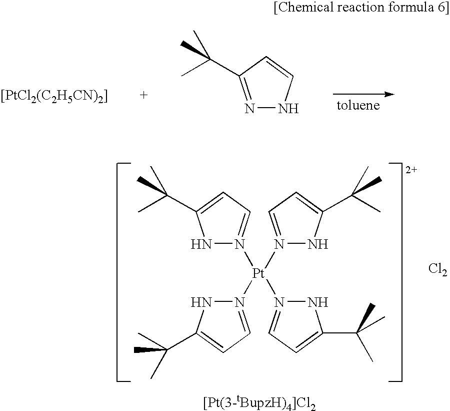

The product exhibits very weak violet luminescence in the solid state upon exposure to UV radiation.

With regard to solubility in a solvent, the compound is readily soluble in chloroform and methylene chloride and soluble in toluene and methanol

The product was identified by the IR and ¹H NMR spectra.

The infrared frequencies are as follows.

IR (KBr): 3388 (br/m), 3103 (br/s), 2965 (br/s), 1562 (m), 1486 (s), 1370 (s), 1300 (s), 1270 (m), 1212 (m), 1135 (s), 990 (m), 823 (s)

The ¹H NMR data are summarized in Table 12.

TABLE 12
¹H NMR of [Pt(3-^{t}BupzH)₄]Cl₂ (CDCl₃, TMS, 300 MHz)

| δ (ppm) | Shape (J/Hz) | Int. | Assighn. |
|---|---|---|---|
| 7.58 | s | 1 | H5 of 3-^{t}BupzH |
| 5.94 | s | 1 | H4 of 3-^{t}BupzH |
| 1.34 | s | 10 | ^{t}Bu of 3-^{t}BupzH |

Next, the precursor complex [Pt(3-^{t}Bupz)₂(3-^{t}BupzH)₂] was synthesized.

Specifically, a solution of the previously synthesized [Pt(3-^{t}BupzH)₄]Cl₂ (193 mg, 0.25 mmol) in methanol (6 ml) was prepared. A solution of KOH (28 mg, 0.52 mmol) in methanol (2 ml) was added dropwise to this methanol solution with stirring at room temperature. The colorless solution was stirred for one hour and then concentrated by an evaporator. The resulting white solid was collected, sequentially washed with a small amount of methanol and water, and dried in vacuo.

The yield was 143 mg (0.21 mmol) (83%).

The synthetic method of [Pt(3-^{t}Bupz)₂(3-^{t}BupzH)₂] is shown in chemical reaction formula 7.

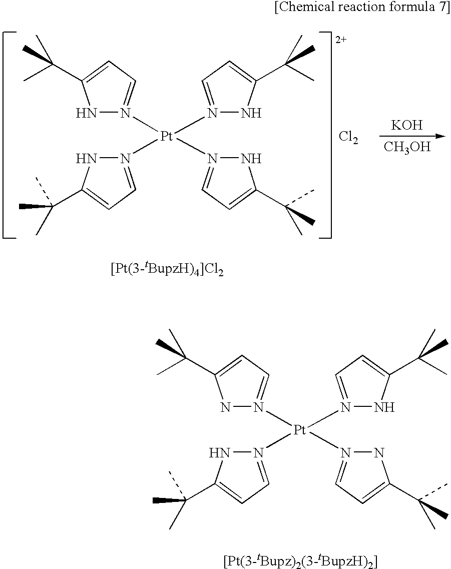

This compound exhibits weak violet luminescence in the solid state upon exposure to UV radiation.

With regard to solubility in a solvent, the compound is readily soluble in chloroform and methylene chloride and soluble in acetone, methanol and hexane.

The product was identified by the IR and ¹H NMR spectra.

The infrared frequencies are as follows.

### IR (KBr): 3613 (w), 2962 (s), 1566 (m), 1474 (s), 1361 (m), 1298 (s), 1241 (m), 1207 (m), 1134 (s), 1049 (m), 755 (s)

The ¹H NMR data are summarized in Table 13.

TABLE 13
¹H NMR of [Pt(3-^{t}Bupz)₂(3-^{t}BupzH)₂] (CDCl₃, TMS, 300 MHz)

| δ (ppm) | Shape (J/Hz) | Int. | Assighn. |
|---|---|---|---|
| 6.99 | s | 1 | NH |
| 6.79 | d | 1 | H5 of 3-^{t}BupzH |
| 5.98 | d | 1 | H4 of 3-^{t}BupzH |
| 1.38 | s | 10 | ^{t}Bu of 3-^{t}BupzH |

Next, the metal complex [Pt₂Ag₄(μ-3-^{t}Bupz)₈] was synthesized from the precursor complex [Pt (3-^{t}Bupz)₂(3-^{t}BupzH)₂].

Specifically, triethylamine (11 mg, 0.11 mmol) and a solution of AgBF₄ (27 mg, 0.14 mmol) in acetonitrile (8 ml) were sequentially added to a suspension of the precursor complex [Pt(3-^{t}Bupz)₂(3-^{t}BupzH)₂] (37 mg, 0.027 mmol) in acetonitrile (10 ml) at room temperature, and the mixture was stirred for two hours. After completion of the reaction, the white solid was collected, washed with acetonitrile, and dried in vacuo. The yield was 43 mg (0.023 mmol) (87%). The synthetic method of [Pt₂Ag₄(μ-3-^{t}Bupz) 8] is shown in chemical reaction formula 8.

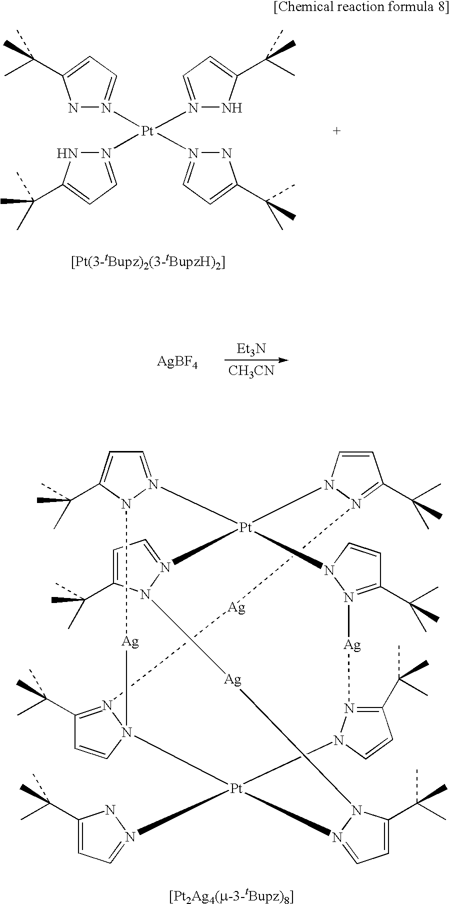

This compound exhibits weak yellow-green luminescence in the solid state upon exposure to UV radiation.

With regard to solubility in a solvent, the compound is readily soluble in chloroform and methylene chloride.

The product was identified by the IR and ¹H NMR spectra.

The infrared frequencies are as follows.

### IR (KBr): 3130 (w), 2963 (s), 1492 (s), 1475 (s), 1344 (s), 1243 (s) 1127 (m), 1073 (s), 1009 (w), 855 (w), 760 (s), 501 (m)

The ¹H NMR data are summarized in Table 14.

TABLE 14
¹H NMR of [Pt₂Ag₄(μ-3-^{t}Bupz)₈] (CDCl₃, TMS, 300 MHz)

| δ (ppm) | Shape (J/Hz) | Int. | Assighn. |
|---|---|---|---|
| 6.51 | t | 1 | H5 of 3-^{t}BupzH |
| 5.89 | t | 1 | H4 of 3-^{t}BupzH |
| 1.14 | s | 10 | ^{t}Bu of 3-^{t}BupzH |

The results of elemental analysis of the product are shown in Table 15 by comparison with calculated values.

TABLE 15
Elemental Analysis of [Pt₂Ag₄(μ-3-^{t}Bupz)₈]

| | Calc. | Found |
|---|---|---|
| C (%) | 37.22 | 36.93 |
| H (%) | 4.91 | 4.65 |
| N (%) | 12.40 | 12.28 |

Next, structures of the precursor complexes and the finally produced metal complexes in Examples 2 and 3 will be described, respectively.

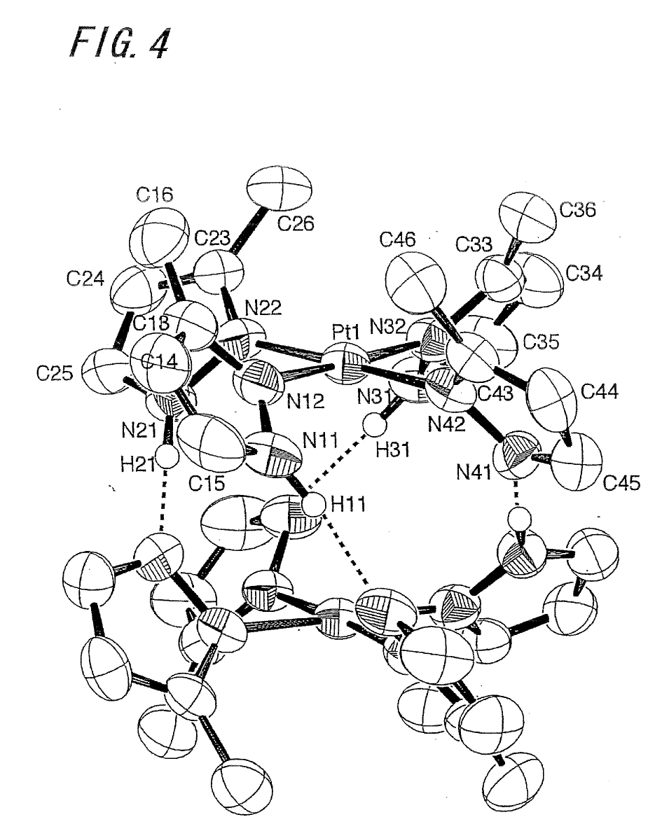

Molecular structures of [{Pt(3-Mepz)₂(3-MepzH)₂}₂], [Pt(3-^{t}Bupz)₂(3-^{t}BupzH)₂] and [Pt₂Ag₄(μ-3-^{t}Bupz)₈] were determined by single crystal X-ray structural analysis. The crystallographic data are shown in Table 16. The molecular structures of the complexes are shown in FIGS. 4 to 6, respectively.

In the molecular structure of [{Pt(3-Mepz)₂(3-MepzH)₂}₂], as shown in the ORTEP diagram of FIG. 4, two molecules of a mononuclear complex {Pt(3-Mepz) 2 (3-MepzH) 2} having two 3-Mepz ligands and two 3-MepzH ligands coordinated to a Pt (II) ion are dimerized by forming strong N—H...N hydrogen bonds between 3-MepzH ligands of one molecule and 3-Mepz ligands of the other molecule. The Pt...Pt distance in the dimer is 3.6843(7) Å.

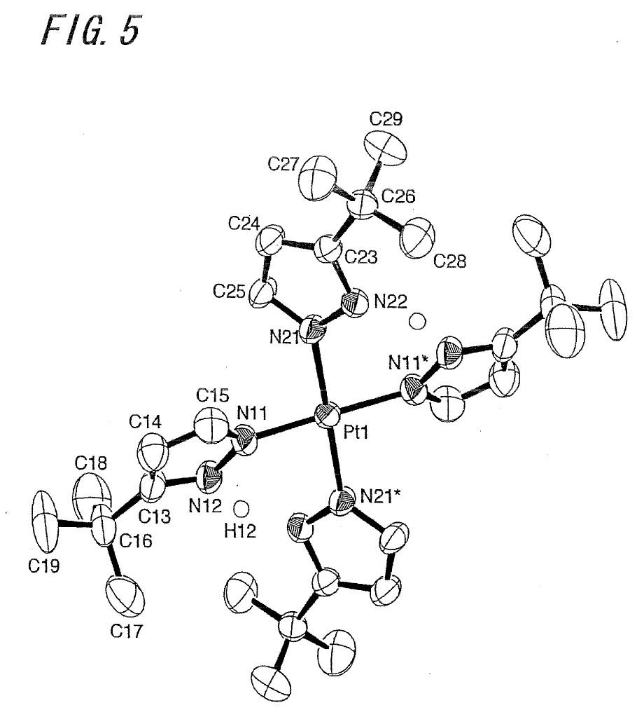

In the molecular structure of [Pt(3-^{t}Bupz)₂(3-^{t}BupzH)₂] as shown in the ORTEP diagram of FIG. 5, two 3-^{t}Bupz ligands and two 3-^{t}BupzH ligands are coordinated to a Pt(II) ion to form a mononuclear complex in which strong N—H...N hydrogen bonds are formed intramolecularly between the 3-^{t}Bupz ligands and the 3-^{t}BupzH ligands. In this case, a dimer complex is not formed intermolecularly.

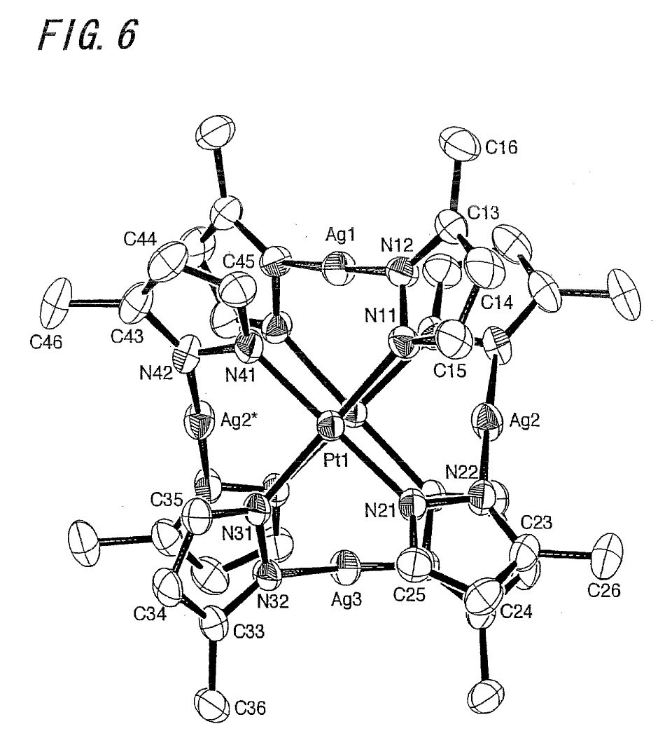

As shown in the ORTEP diagram of FIG. 6, the molecular structure of [Pt₂Ag₄(μ-3-^{t}Bupz)₈] is similar to that of [Pt₂Ag₄(μ-dmpz)₈]. All substituents groups in the 3-^{t}Bupz ligands are located on the C atoms adjacent to the N atoms coordinating to Ag atoms. [Pt₂Ag₄(μ-3-^{t}Bupz)₈] has an idealized 4-fold axis passing through the two Pt atoms and two different sets of 2-fold axes are normal to the 4-fold rotation axis. The Pt...Pt distance in [Pt₂Ag₄(μ-3-^{t}Bupz)₈] is 4.4988(2) Å and Pt...Ag distances are ranging from 3.4382(3) to 3.4709(3) Å.

TABLE 16
Crystallographic data of [{Pt(3-Mepz)₂(3-MepzH)₂}₂],
[Pt(3-^{t}Bupz)₂(3-^{t}BupzH)₂] and [Pt₂Ag₄(μ-3-^{t}Bupz)₈]

| | [{Pt(3-Mepz)₂(3-MepzH)₂}₂] | [Pt(3-^{t}Bupz)₂(3-^{t}BupzH)₂] | [Pt₂Ag₄(μ-3-^{t}Bupz)₈] |
|---|---|---|---|
| empirical formula | C₃₂H₄₄N₁₆Pt₂ | C₂₈H₆₆N₈Pt | C₃₆H₈₈Ag₄N₁₆Pt₂ |
| fw | 1042.99 | 689.81 | 1807.07 |
| T, K | 296 | 296 | 296 |
| λ, Å | 0.71070 | 0.71070 | 0.71070 |
| cryst syst | monoclinic | monoclinic | monoclinic |
| space group | C2/c (15) | P2₁/a (14) | C2/c (15) |
| a, Å | 24.276 (4) | 11.0918 (7) | 28.270 (2) |
| b, Å | 9.770 (1) | 10.6626 (5) | 14.3273 (5) |
| c, Å | 18.707 (3) | 13.868 (1) | 18.919 (1) |
| α, deg | 90 | 90 | 90 |
| β, deg | 124.348 (2) | 105.0977 (9) | 116.4684 (7) |
| γ, deg | 90 | 90 | 90 |
| V, Å³ | 3663.1 (9) | 1583.5 (2) | 6859.5 (6) |
| Z | 4 | 2 | 4 |
| ρ_{calcd}, Mg m⁻³ | 1.891 | 1.447 | 1.750 |
| μ(Mo Kα), mm⁻¹ | 7.647 | 4.442 | 5.208 |
| no. of unique rflns | 3938 (Rᵢₙₜ = 0.029) | 3406 (Rᵢₙₜ = 0.026) | 7689 (Rᵢₙₜ = 0.032) |
| data/restraints/params | 3938/0/226 | 3404/0/173 | 7687/0/353 |
| final R indices [I > 2σ (I)] | R1 = 0.042 | R1 = 0.030 | R1 = 0.028 |
| R indices (all data) | R = 0.068, R_{w} = 0.112 | R = 0.062, R_{w} = 0.076 | R = 0.055, R_{w} = 0.070 |
| GOF | 1.03 | 0.96 | 0.79 |

### Example 4

As one of the first metal complexes of the present invention, [Pt₂Cu(μ-dmpz)₈] was synthesized by using [{Pt(dmpz)₂(dmpzH)₂}₂], which was also used in Example 1 as the precursor complex.

Details of the synthetic method of the metal complex will be described below.

First, the precursor complex [{Pt(dmpz)₂(dmpzH)₂}₂] was synthesized in the same manner as in Example 1.

Next, the metal complex [Pt₂Cu₄(μ-dmpz)₈] was synthesized from the precursor complex [{Pt(dmpz)₂(dmpzH)₂}₂].

Specifically, triethylamine (18 mg, 0.18 mmol) and a solution of [Cu(CH₃CN)₄]BF₄ (56.6 mg, 0.18 mmol) in methylene chloride (10 ml) were sequentially added to a solution of [{Pt(dmpz)₂(dmpzH)₂}₂] (51.7 mg, 0.045 mmol) in methylene chloride (20 ml) under an Ar atmosphere, and the mixture was stirred for two hours. Thereafter, the solution was filtered, and the filtrate was concentrated to dryness by an evaporator. A small amount of acetonitrile was added to the solid, and the mixture was filtered. The solid was further washed with acetonitrile and then dried in vacuo. The yield was 46.4 mg (0.033 mmol) (73.7%). This metal complex was recrystallized from chloroform/toluene. The synthetic method of [Pt₂Cu₄(μ-dmpz)₈] is shown in chemical reaction formula 9.

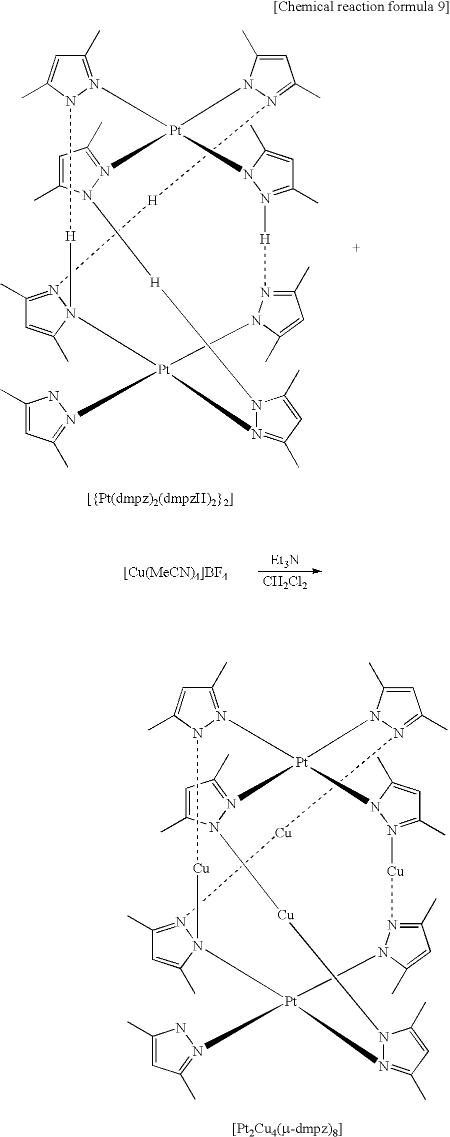

This complex exhibits orange luminescence in the solid state upon exposure to UV radiation.

With regard to solubility in a solvent, the compound is soluble in chloroform and methylene chloride, slightly soluble in ether and acetone, and poorly soluble in acetonitrile, methanol and toluene.

The product was identified by the IR and ¹H NMR spectra.

The infrared frequencies are as follows.

### IR (KBr): 3112 (w), 2918 (m), 2853 (w), 1530 (s), 1420 (s), 1357 (m), 1149 (w), 1036 (w), 980 (w), 762 (s), 652 (w), 591 (w), 473 (w), 406 (w), 350 (w), 326 (w)

The ¹H NMR data are summarized in Table 17.

TABLE 17
¹H NMR of [Pt₂Cu₄(μ-dmpz)₈] (CDCl₃, TMS, 300 MHz)

| δ (ppm) | Shape (J/Hz) | Int. | Assighn. |
|---|---|---|---|
| 5.62 | s | 1 | H4 of dmpzH |
| 1.84 | s | 3 | 5-CH₃ of dmpzH |
| 1.77 | s | 3 | 3-CH₃ of dmpzH |

The results of elemental analysis of the product are shown in Table 18 by comparison with calculated values.

TABLE 18
Elemental Analysis of [Pt₂Cu₄(μ-dmpz)₈]

| | Calc. | Found |
|---|---|---|
| C (%) | 34.19 | 34.54 |
| H (%) | 4.02 | 3.83 |
| N (%) | 15.95 | 15.81 |

### Example 5

As one of the first metal complex of the present invention, [Pt₂Cu₄(μ-3-^{t}Bupz)₈] was synthesized by using [Pt(3-^{t}Bupz)₂(3-^{t}BupzH)₂], which was also used in Example 3 as the precursor complex.

Details of the synthetic method of the metal complex will be described below.

First, the precursor complex [Pt(3-^{t}Bupz)₂(3-^{t}BupzH)₂] was synthesized in the same manner as in Example 3.

Next, the metal complex [Pt₂Cu₄(μ-3-^{t}Bupz)₈] was synthesized from the precursor complex [Pt(3-^{t}Bupz)₂(3-^{t}BupzH)₂].

Specifically, triethylamine (18 mg, 0.18 mmol) and a solution of [Cu (CH₃CN)₄]BF₄ (51 mg, 0.16 mmol) in methylene chloride (15 ml) were sequentially added to a solution of [Pt(3-^{t}Bupz)₂(3-^{t}BupzH)₂] (54 mg, 0.077 mmol) in methylene chloride (5 ml) at room temperature, and the mixture was stirred under an Ar atmosphere for five hours. After completion of the reaction, a small amount of the precipitated salt was filtered off, and the filtrate was concentrated slowly under air to yield crystals. The yield was 34 mg (0.02 mmol) (54%). The synthetic method of [Pt₂Cu₄(μ-3-^{t}Bupz)₈] is shown in chemical reaction formula 10.

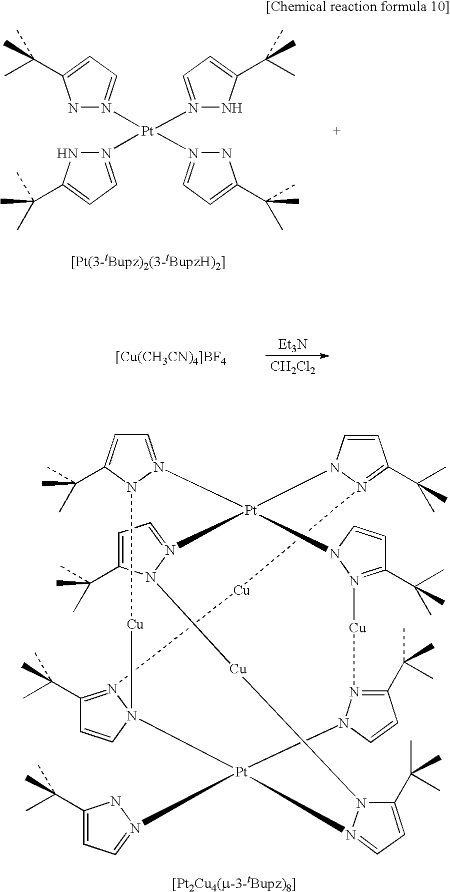

Emission from this compound was not observed by the naked eye upon exposure to UV radiation.

With regard to solubility in a solvent, the compound is soluble in chloroform and methylene chloride.

The product was identified by the IR and ¹H NMR spectra.

The infrared frequencies are as follows.

### IR (KBr): 3137 (w), 2964 (s), 1494 (s), 1478 (s), 1349 (s), 1242 (s), 1124 (s), 1079 (s), 1013 (m), 857 (m), 761 (s), 724 (m), 646 (m), 510 (m)

The ¹H NMR data are summarized in Table 19.

TABLE 19
¹H NMR of [Pt₂Cu₄(μ-3-^{t}Bupz)₈] (CDCl₃, TMS, 300 MHz)

| δ (ppm) | Shape (J/Hz) | Int. | Assighn. |
|---|---|---|---|
| 6.42 | d | 1 | H5 of 3-^{t}BupzH |
| 5.84 | d | 1 | H4 of 3-^{t}BupzH |
| 1.16 | s | 10 | ^{t}Bu of 3-^{t}BupzH |

The results of elemental analysis of the product are shown in Table 20 by comparison with calculated values.

TABLE 20
Elemental Analysis of [Pt₂Cu₄(μ-3-^{t}Bupz)₈]

| | Calc. | Found |
|---|---|---|
| C (%) | 41.27 | 41.25 |
| H (%) | 5.44 | 5.22 |
| N (%) | 13.75 | 13.83 |

Next, structures of the finally produced metal complexes in Examples 4 and 5 will be described, respectively.

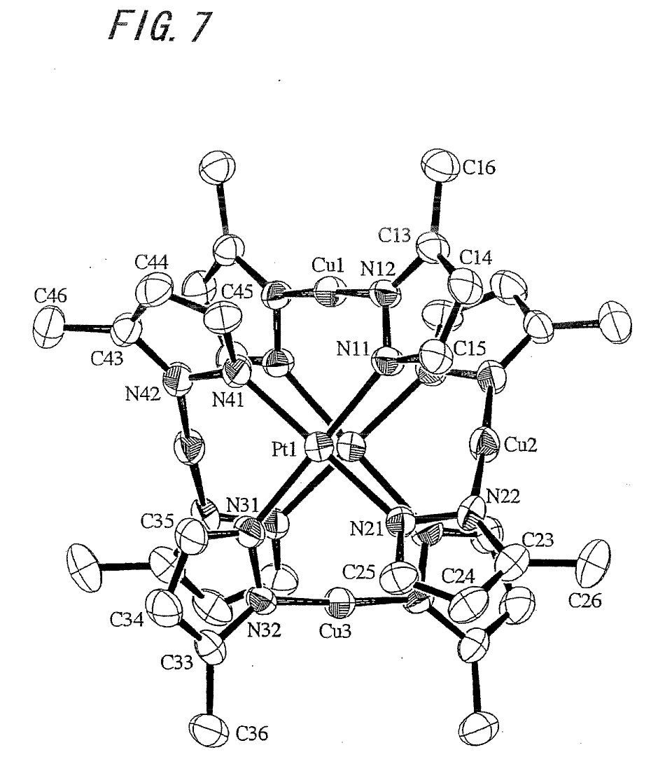

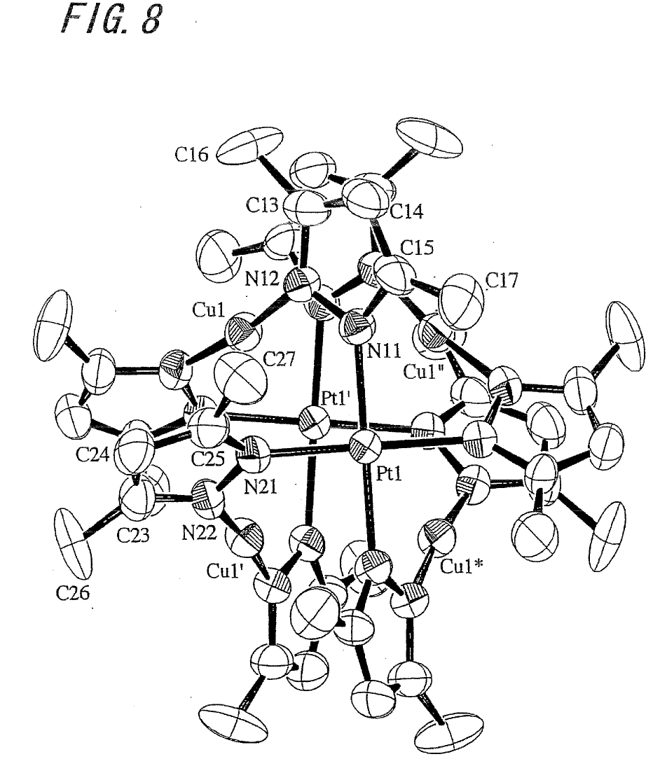

Molecular structures of [Pt₂Cu₄(μ-3-^{t}Bupz)₈] and [Pt₂Cu₄(μ-dmpz)₈] were determined by single crystal X-ray structural analysis. The crystallographic data are shown in Table 21. The molecular structures of the compounds are shown in FIGS. 7 and 8, respectively.

As shown in the ORTEP diagram of FIG. 7, the molecular structure of [Pt₂Cu₄(μ-3-^{t}Bupz)₈] is very similar to that of [Pt₂Ag₄ (μ-3-^{t}Bupz)₈]. All substituent groups in the 3-^{t}Bupz ligands are located on the C atoms adjacent to the N atoms coordinating to Cu atoms. [Pt₂Cu₄(μ-3-^{t}Bupz)₈] has an idealized 4-fold axis passing through the two Pt atoms and two different sets of 2-fold axes are normal to the 4-fold rotation axis. The Pt...Pt distance in [Pt₂Cu₄(μ-3-^{t}Bupz)₈] is 3.8626(3) Å and the Pt...Cu distances are ranging from 3.3586(7) to 3.3890(7) Å.

As shown in the ORTEP diagram of FIG. 8, the molecular structure of [Pt₂Cu₄(μ-dmpz)₈] is very similar to that of [Pt₂Ag₄(μ-dmpz)₈]. [Pt₂Cu₄(μ-dmpz)₈] has a crystallographically imposed 4-fold axis passing through the two Pt atoms and two different sets of crystallographically imposed 2-fold axes are normal to the 4-fold rotation axis. The Pt...Pt distance in [Pt₂Cu₄(μ-dmpz)₈] is 4.6567(5) Å and the Pt—Cu distances are ranging from 3.365(3) and 3.367(3) Å.

TABLE 21
Crystallographic data of
[Pt₂Cu₄(μ-3-^{t}Bupz)₈] and [Pt₂Cu₄(μ-dmpz)₈]

| | [Pt₂Cu₄(μ-3-t-Bupz)₈] | [Pt₂Cu₄(μ-dmpz)₈] |
|---|---|---|
| empirical formula | C₅₆H₈₈Cu₄N₁₆Pt₂ | C₄₀H₅₆Cu₄N₁₆Pt₂ |
| fw | 1629.78 | 1405.35 |
| T, K | 296 | 296 |
| λ, Å | 0.71070 | 0.71070 |
| cryst syst | monoclinic | orthorhombic |
| space group | C2/c (15) | I222 (23) |
| a, Å | 27.880 (2) | 13.4240 (7) |
| b, Å | 14.620 (1) | 13.4260 (5) |
| c, Å | 18.047 (1) | 13.7605 (5) |
| α, deg | 90 | 90 |
| β, deg | 114.3563 (2) | 90 |
| γ, deg | 90 | 90 |
| V, Å³ | 6701.2 (9) | 2480.1 (2) |
| Z | 4 | 2 |
| ρ_{calcd}, Mg m⁻³ | 1.615 | 1.882 |
| μ(Mo Kα), mm⁻¹ | 5.435 | 7.327 |
| no. of unique rflns | 7636 (Rᵢₙₜ = 0.038) | 1589 (Rᵢₙₜ = 0.019) |
| data/restraints/params | 7636/0/353 | 1589/0/142 |
| final R indices | R1 = 0.034 | R1 = 0.027 |
| [I > 2σ (I)] | | |
| R indices (all data) | R = 0.055, R_{w} = 0.093 | R = 0.057, R_{w} = 0.070 |
| GOF | 0.95 | 0.97 |
| GOF | 0.95 | 0.97 |

Next, photochemical properties of [Pt₂Ag₄(μ-3-Mepz)₈], [Pt₂Ag₄(μ-3-^{t}Bupz)₈] and [Pt₂Cu₄(μ-dmpz)₈] will be described.

The emission quantum yield (in dichloromethane) was measured for each metal complex. The emission quantum yield was determined by using a solution of [Pt₂Ag₄(μ-dmpz)₈] in CH₂Cl₂ (Φ=0.51, under degassed conditions) as a reference (standard material). All sample solutions were deoxygenated with a stream of argon prior to the measurement. The results are summarized in Table 22.

Relatively strong green luminescence was observed for [Pt₂Ag₄(μ-3-Mepz)₈] in solution. However, [Pt₂Ag₄(μ-3-^{t}Bupz)₈] exhibits extremely weak luminescence. [Pt₂Cu₄(μ-dmpz)₈] exhibits luminescence in near infrared region (λₘₐₓ=820 nm).

Emission intensity in a crystalline state was also measured. All measurements were performed with equal amounts of the samples at the same excitation wavelength (270 nm). Emission intensity is summarized in Table 22.

It is presumed that the order of emission intensity in the solid state reflects that of the emission quantum yield in solution.

TABLE 22
Emission properties of [Pt₂M₄(μ-L)₈]
(M = Ag, Cu; L = dmpz, 3-Mepz, 3-¹Bupz) in crystal and CH₂Cl₂

| | Crystal | | In CH₂Cl₂ | |
|---|---|---|---|---|
| | λₘₐₓ/nm | Em. Intensity | λₘₐₓ/nm | Φ |
| [Pt₂Ag₄(μ-dmpz)₈] | 497 | 2755 | 528 | 0.51 |
| [Pt₂Ag₄(μ-3-Mepz)₈] | 482 | 820 | 546 | 0.14 |
| [Pt₂Ag₄(μ-3-^{t}Bupz)₈] | ~525 | 10 | 537 | ~5 × 10⁻⁵ |
| [Pt₂(dmpz)₄(dmpzH)₄] | ~556 | 50 | 600 | 0.001 |
| [Pt₂Cu₄(μ-dmpz)₈] | 625 | 67 | 820 | 0.042 |

The emission lifetimes of [Pt₂Ag₄(μ-3-Mepz)₈] and [Pt₂Cu₄(μ-dmpz)₈] in dichloromethane were also measured. The sample solutions were deoxygenated with a stream of argon over 30 minutes. Detection was performed by using the fourth-harmonic generation of a Nd⁺-YAG Laser (266 nm, repetition 10 Hz, 10 mJ/pulse) as an excitation light source with a streak camera (Hamamatsu Photonics Inc. C4334). Emission decay curves of [Pt₂Ag₄(μ-3-Mepz) 8] and [Pt₂Cu₄(μ-dmpz)₈] were measured in a CH₂Cl₂ solution. Each emission decay curve was analyzed by the equation (I(t)=A₁exp(−t/τ₁)+A₂exp(−t/τ₂)) using the nonlinear least-squares method. The results are listed in Table 23.

TABLE 23
Emission decay parameters of [Pt₂Ag₄(μ-3-Mepz)₈]
and [Pt₂Cu₄(μ-dmpz)₈] in CH₂Cl₂

| | τ₁/μs | A₁ | τ₂/μs | A₂ |
|---|---|---|---|---|
| [Pt₂Ag₄(μ-3-Mepz)₈] | 1.3 | 0.54 | 8.1 | 0.46 |
| [Pt₂Cu₄(μ-dmpz)₈] | | | 11.9 | 1.00 |

[Pt₂Ag₄(μ-3-Mepz)₈] can be analyzed by a double exponential function. The ratio of the short lifetime component (1.3 μs) to the long lifetime component (8.1 μs) was approximately 1:1.

In contrast, [Pt₂Cu₄(μ-dmpz)₈] can be analyzed by a single exponential function.

### Example 6

A metal complex [PtCl(dppz)(dppzH)₂] was synthesized as a precursor complex, and [Pt₂Ag₄(μ-Cl)₂(μ-dppz)₆], which is one of the second metal complex of the present invention, was synthesized by using this precursor complex.

Details of the synthetic method of the metal complex will be described below.

First, the intermediate product for synthesizing the precursor complex [PtCl(dppz)(dppzH)₂] was prepared.

Specifically, a solution of dmpzH (136 mg, 0.62 mmol) in toluene (15 ml) was added to a suspension of [PtCl₂(C₂H₅CN)₂] (60 mg, 0.16 mmol) in toluene (5 ml), and the mixture was refluxed overnight under an Ar atmosphere. The resulting white yellow precipitate was collected, sequentially washed with toluene, hexane and diethyl ether, and then dried in vacuo. The yield was 137 mg. Chemical reaction to yield this white yellow solid is shown in chemical reaction formula 11.

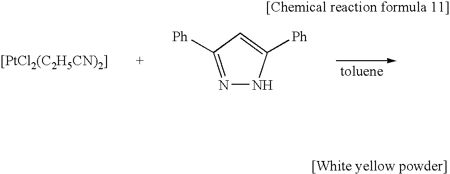

This white yellow solid was identified by the IR spectrum.

The infrared frequencies are as follows.

IR (KBr): 3063 (br), 1572 (s), 1461 (s), 1271 (m), 1189 (m), 1107 (w), 1078 (m), 756 (s), 684 (s), 481 (w), 342 (w)

With regard to solubility in a solvent, the compound is slightly soluble in chloroform, dichloromethane, acetonitrile and methanol.

Next, the precursor complex [PtCl(dppz)(dppzH)₂] was synthesized from this white yellow solid.

Specifically, a solution of KOH (35 mg, 0.62 mmol) in methanol (2 ml) was added dropwise to a suspension of the white yellow solid (198 mg, 0.17 mmol) in methanol (18 ml) with stirring at room temperature, so that the solution immediately changed to a white suspension. After stirring for one hour, the formed white solid was collected, sequentially washed with methanol and water, and then dried in vacuo. The yield was (177 mg, 0.20 mmol). The synthetic method of [PtCl(dppz)(dppzH)₂] is shown in chemical reaction formula 12.

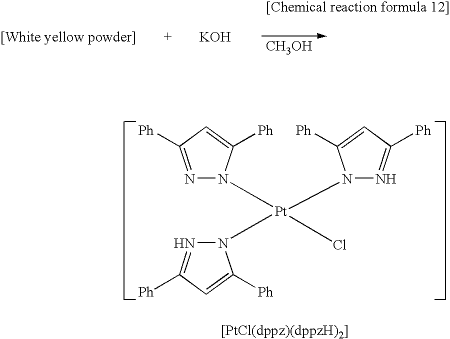

The metal complex was recrystallized from dichloromethane/methanol to yield a single crystal.

This complex exhibits weak pale orange luminescence in the solid state upon exposure to UV radiation.

With regard to solubility in a solvent, the compound is readily soluble in chloroform, dichloromethane and acetone and soluble in benzene, toluene and acetonitrile.

The product was identified by the IR and ¹H NMR spectra.

The infrared frequencies are as follows.

### IR (KBr): 3450 (w), 3111 (w), 3064 (w), 1603 (m), 1573 (m), 1464 (s), 1274 (w), 1212 (w), 1072 (m), 911 (w), 757 (s), 692 (s), 344 (w)

The ¹H NMR data are summarized in Table 24.

TABLE 24
¹H NMR of [PtCl(dppz)(dppzH)₂] (CDCl₃, TMS, 300 MHz)

| δ (ppm) | Shape | Int. | Assign. |
|---|---|---|---|
| 8.12 | d | 4 | Ph of dppzH |
| 7.94 | d | 2 | Ph of dppz |
| 7.51-7.04 | m | 24 | Ph of dppz & dppzH |
| 6.45 | s | 2 | H4 of dppzH |
| 6.24 | s | 1 | H4 of dppz |

Next, the metal complex [Pt₂Ag₄(μ-Cl)₂(μ-dppz)₆] was synthesized from the precursor complex [PtCl(dppz)(dppzH)₂].

Specifically, triethylamine (18 mg, 0.18 mmol) and a solution of AgBF₄ (26 mg, 0.13 mmol) in acetonitrile (10 ml) were sequentially added to a suspension of the precursor complex [PtCl (dppz)(dppzH)₂] (54 mg, 0.06 mmol) in methanol (10 ml) at room temperature, and the mixture was stirred for two hours. Thereafter, the white solid was collected, washed with acetonitrile, and then dried in vacuo. The yield was 59 mg (0.027 mmol) (89%). The synthetic method of [Pt₂Ag₄(μ-Cl)₂(μ-dppz)₆] is shown in chemical reaction formula 13.

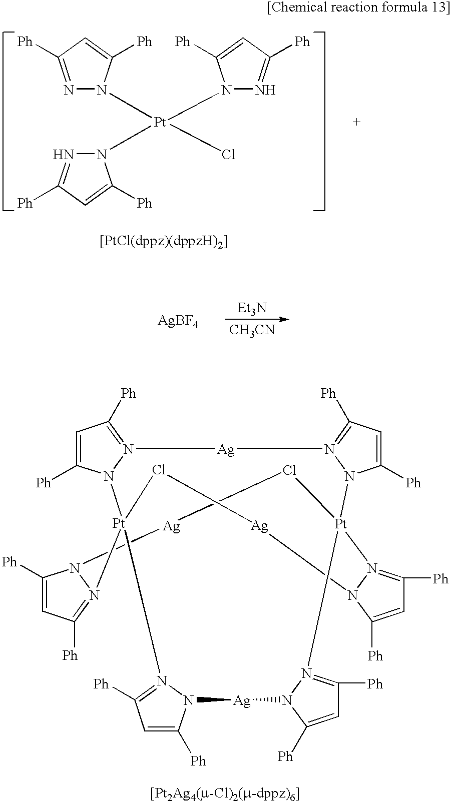

This metal complex was recrystallized from dichloromethane/methanol to yield a single crystal.

This complex exhibited bright red-orange luminescence in the solid state and weak green luminescence in solution, respectively, upon exposure to UV radiation.

With regard to solubility in a solvent, the compound is readily soluble in chloroform and dichloromethane, soluble in benzene and toluene, and slightly soluble in acetonitrile.

The product was identified by the IR spectrum. The infrared frequencies are as follows.

### IR (KBr): 3061 (w), 1603 (m), 1472 (s), 1403 (m), 1334 (w), 1279 (w), 1111 (w), 1072 (w) 912 (w), 754 (s) 696 (s), 304 (w)

The results of elemental analysis of the product are shown in Table 25 by comparison with calculated values

TABLE 25
Elemental Analysis of [Pt₂Ag₄Cl₂(dppz)₆]

| | Calc. | Found. | Δ |
|---|---|---|---|
| C (%) | 48.95 | 48.84 | −0.11 |
| H (%) | 3.01 | 2.90 | −0.11 |
| N (%) | 7.61 | 7.64 | 0.03 |

Structures of the precursor complex and the finally produced metal complex will be described, respectively.

Molecular structures of [PtCl(dppz)(dppzH)₂] and [Pt₂Ag₄(μ-Cl)₂(μ-dppz)₆] were determined by single crystal X-ray structural analysis. The crystallographic data are shown in Table 26.

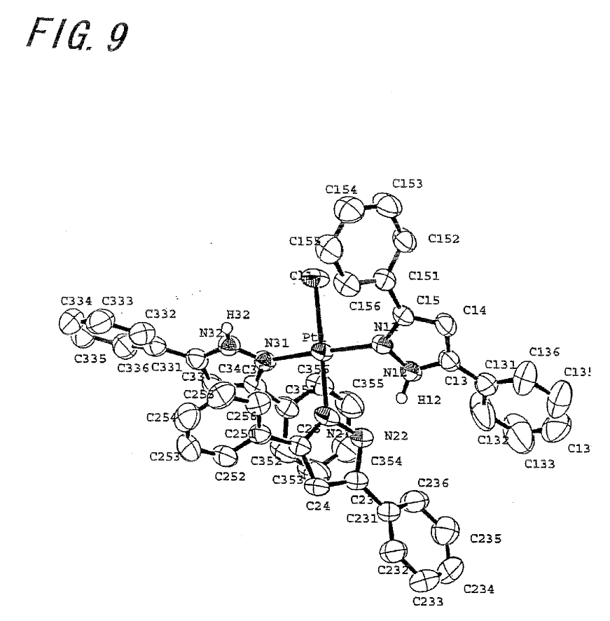

TABLE 26
Crystallographic data of
[PtCl(dppz)(dppzH)₂] and [Pt₂Ag₄(μ-Cl)₂(μ-dppz)₆]

| | [PtCl(dppz)(dppzH)₂]═CH₃OH | [Pt₂Ag₄(μ-Cl)₂(μ-dppz)₆] |
|---|---|---|
| empirical formula | C₄₆H₃₉ClN₆OPt | C₉₀H₆₆Cl₂N₁₆Ag₄Pt₂ |
| fw | 922.40 | 2264.18 |
| T, K | 296 | 296 |
| λ, Å | 0.71069 | 0.71069 |
| cryst syst | monoclinic | monoclinic |
| space group | p2₁/n (14) | P2₁/n (14) |
| a, Å | 15.905 (6) | 15.229 (4) |
| b, Å | 9.712 (4) | 29.469 (7) |
| c, Å | 27.34 (1) | 19.231 (4) |
| α, deg | 90 | 90 |
| β, deg | 105.571 (2) | 92.2363 (8) |
| γ, deg | 90 | 90 |
| V, Å³ | 4068.2 (2) | 8624.0 (2) |
| Z | 4 | 4 |
| ρ_{calcd}, Mg m⁻³ | 1.506 | 1.744 |
| μ (Mo Kα), mm⁻¹ | 3.544 | 4.223 |
| no. of unique rflns | 9179 (Rᵢₙₜ = 0.029) | 18976 (Rᵢₙₜ = 0.030) |
| data/restraints/params | 9175/0/508 | 18976/0/991 |
| final R indices [I > 2σ (I)] | R1 = 0.032 | R1 = 0.050 |
| R indices (all data) | R = 0.043, R_{w} = 0.075 | R = 0.081, R_{w} = 0.161 |
| GOF | 0.92 | 1.33 |

[PtCl(dppz)(dppzH)₂] crystallized with a methanol molecule as a crystal solvent. As shown in the ORTEP diagram of FIG. 9, one Cl⁻ ion, one dppz ligand and two dppzH ligands coordinated to the Pt(II) ion in the molecular structure. One of the two dppzH ligands forms a hydrogen bonding (N12-H12...N22) together with the dppz ligand.

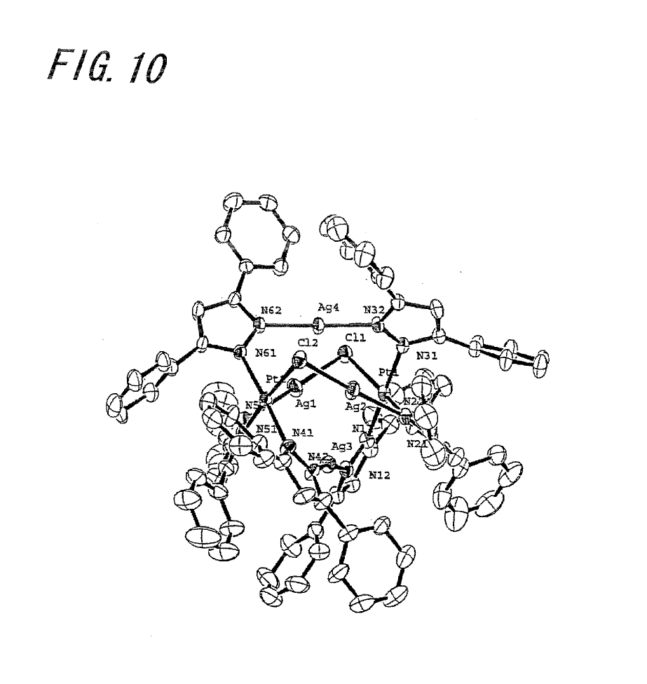

As shown in the ORTEP diagram of FIG. 10, in the molecular structure of [Pt₂Ag₄(μ-Cl)₂(μ-dppz)₆], four protons (H⁺) in total are liberated from two molecules of [PtCl(dppz)(dppzH)₂] and four Ag⁺ ions are incorporated in the molecules to form one molecule of [Pt₂Ag₄(μ-Cl)₂(μ-dppz)₆]. One Cl⁻ ion and three dppz ligands coordinates to each Pt(II) ion, and each Ag⁺ ion is located between the two dppz ligands or between the Cl⁻ ion and the dppz ligand. The complex molecule has an idealized 2-fold axis passing through Ag3 atom and Ag4 atom and the complex also has an idealized mirror plane defined by Pt1, Pt2, Ag3 and Ag4.

The Pt...Pt distance in [Pt₂Ag₄(μ-Cl)₂(μ-dppz)₆] is 5.2873(5) Å. The Pt...Ag distances are ranging from 3.0816(8) to 3.6535(7) Å, and the proximate Ag...Ag distances are ranging from 2.936(1) to 4.725(1) Å.

[Pt₂Ag₄ (μ-Cl)₂(μ-dppz)₆] exhibited bright orange luminescence in the solid state upon exposure to UV radiation at 300 nm or 350 nm, and its emission spectrum had the local maximum at 652 nm. The complex exhibited broad emission spectrum around 450 nm to 600 nm in dichloromethane, but the shape of the spectrum changed gradually with increasing the emission intensity.

As described above, the metal complexes of the present invention have potential for industrial applications such as light-emitting device and display.

### DESCRIPTION OF SYMBOLS

1 Substrate
2 Anode
3 Hole injection layer
4 Hole transport layer
5 Light-emitting layer
6 Electron transport layer
7 Electron injection layer
8 Cathode

1. A metal complex comprising the following composition: [(Pt^{II})₂(M^{I})₄(L)₈]
wherein M^{I} is H⁺, Ag^{I}, Au^{I} or Cu^{I}, and L is a compound represented by the following chemical formula: 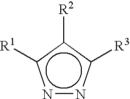
wherein R¹, R² and R³ are independently a hydrogen atom, a chlorine atom, a bromine atom, an iodine atom, a hydroxyl group, a phenyl group, a trifluoromethylphenyl group, a pentafluorophenyl group, a naphthyl group, a methyl group, an ethyl group, an i-propyl group, a t-butyl group, a trifluoromethyl group, a hydroxymethyl group or a hydroxyethyl group, provided that at least one of R¹, R² and R³ is not a hydrogen atom.

2. A light-emitting device comprising a light-emitting layer,
characterized in that the light-emitting layer includes the metal complex according to claim 1.

3. A display comprising a light-emitting device as a component, the light-emitting device having a light-emitting layer,
characterized in that the light-emitting layer includes the metal complex according to claim 1.

4. A metal complex comprising the following composition: [(Pt^{II})₂ (M^{I})₄ (X)₂(L)₆]
wherein M^{I} is Ag^{I}, Au^{I} or Cu^{I}, X is Cl⁻, Br⁻ or I⁻, and L is a compound represented by the following chemical formula: 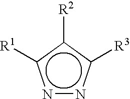
wherein R¹, R² and R³ are independently a hydrogen atom, a chlorine atom, a bromine atom, an iodine atom, a hydroxyl group, a phenyl group, a trifluoromethylphenyl group, a pentafluorophenyl group, a naphthyl group, a methyl group, an ethyl group, an i-propyl group, a t-butyl group, a trifluoromethyl group, a hydroxymethyl group or a hydroxyethyl group, provided that at least one of R¹, R² and R³ is not a hydrogen atom.

5. A light-emitting device comprising a light-emitting layer,
characterized in that the light-emitting layer includes the metal complex according to claim 4.

6. A display comprising a light-emitting device as a component, the light-emitting device having a light-emitting layer,
characterized in that the light-emitting layer includes the metal complex according to claim 4.
